(19) United States
(12) Patent Application Publication
(10) Pub. No.: US 2025/0129143 A1
(43) Pub. Date: Apr. 24, 2025

(54) ANTI-UBIQUITIN SYNTHETIC ANTIBODY

(71) Applicants: TOKYO METROPOLITAN INSTITUTE OF MEDICAL SCIENCE, Tokyo (JP); NATIONAL UNIVERSITY CORPORATION TOKAI NATIONAL HIGHER EDUCATION AND RESEARCH SYSTEM, Aichi (JP); KYOTO UNIVERSITY, Kyoto (JP)

(72) Inventors: Yasushi SAEKI, Tokyo (JP); Akinori ENDO, Tokyo (JP); Yasuko KAWASE, Tokyo (JP); Hiroshi MURAKAMI, Aichi (JP); Gosuke HAYASHI, Aichi (JP); Atsushi SUGIHARA, Aichi (JP); Shuya FUKAI, Kyoto (JP); Kei OKATSU, Kyoto (JP)

(21) Appl. No.: 18/833,769

(22) PCT Filed: Jan. 27, 2023
(86) PCT No.: PCT/JP2023/002596

§ 371 Date: Jul. 26, 2024

(30) Foreign Application Priority Data
Jan. 27, 2022 (JP) 2022-010877

(51) Int. Cl. C07K 16/18; C07K 14/78; G01N 33/68

(52) U.S. Cl. CPC C07K 16/18; C07K 14/78; G01N 33/6872; C07K 2317/92; C07K 2319/30

(57) ABSTRACT

A synthetic anti-ubiquitin antibody, comprising a fibronectin type III polypeptide monobody.

### TECHNICAL FIELD

The present invention relates to a synthetic anti-ubiquitin antibody comprising a fibronectin type III polypeptide.

### BACKGROUND ART

Ubiquitin is a small globular protein consisting of 76 amino acids that is highly conserved in eukaryotes, and when the C-terminal Gly residue (Gly76) is covalently conjugated to the Lys residue side chain of a target protein, the ubiquitin functions as a modifier that regulates the fate of the target protein in various ways (FIG. 1) (Non Patent Literatures 1 and 2). The ubiquitination is carried out by the enzymatic cascade of ubiquitin-activating enzyme E1, ubiquitin-conjugating enzyme E2 and ubiquitin ligase E3, and about 600 types of E3 enzymes are responsible for substrate selectivity.

Recent large-scale analyses have revealed that 9,200 types of proteins, which correspond to approximately 40% of the human proteome, are ubiquitinated, clearly indicating that ubiquitination is a large-scale post-translational modification comparable to phosphorylation. The greatest feature of ubiquitin modification is not only monoubiquitination, in which a single molecule of ubiquitin binds to a substrate, but also further ubiquitination and elongation of ubiquitins on the substrate to form a bead-like polymer (polyubiquitin chain).

It has been found that seven Lys residues (K6, K11, K27, K29, K33, K48, and K63) and an initiating Met residue (M1) of ubiquitin are used for polymer formation. It has been known that, among these 8 types of ubiquitin chains, the K48 chain functions as a proteasomal degradation signal, the K63 chain functions as a signal for membrane protein transport, DNA repair and ubiquitin-selective autophagy, and the M1 chain regulates inflammatory signals by NF-κB. The cellular functions regulated by other ubiquitin chains (atypical ubiquitin chains) are not fully understood, but new findings such as the involvement of the K6 chain in mitophagy and the involvement of the K27 chain in innate immune response are accumulating one after another. Thus, since various structures of polyubiquitin chains specify their functions, the functional information contained in the higher-order structures of ubiquitin modification has come to be called “ubiquitin code” (Non Patent Literature 3).

Although IgG-type monoclonal antibodies against ubiquitin (anti-ubiquitin antibodies) are commonly used to detect ubiquitinated proteins, they are known to have low affinity for ubiquitin monomers and to have different reactivity depending on the types of ubiquitin chains (Non Patent Literature 4). For example, the anti-ubiquitin antibody (clone name: FK2) of Millipore reacts more strongly with the M1 chain than with the K48 chain or the K63 chain, while the anti-ubiquitin antibody (clone name: P4D1) of Cell Signaling Technology reacts more strongly with the K63 chain than with the K48 chain (Non Patent Literature 4).

On the other hand, TUBE (Tandem ubiquitin binding entity) has been developed as an artificial probe to capture and detect polyubiquitinated substrates (Non Patent Literature 5). TUBE is an artificial protein, in which 4 ubiquitin-binding domains (UBA domains) of the ubiquitin-binding protein UBQLN1 or RAD23A are connected in tandem, and the binding affinity (Kd) of TUBE to K48-linked ubiquitin chain (4-mer) or K63 ubiquitin chain (4-mer) is 1 to 9 nM. However, TUBE can only weakly interact with a ubiquitin monomer at about 30 to 66 uM (Non Patent Literature 5).

In addition, IgG-type antibodies, TUBE, nanobodies and the like that are specific for the K48 and K63 chains have been developed, and studies regarding the ubiquitin code are in progress (Non Patent Literature 6). However, the development of panubiquitin probes that can capture and detect all ubiquitinated substrates including monoubiquitinated substrates has been delayed.

Meanwhile, a fibronectin type III polypeptide monobody is known as an artificial probe for capturing and detecting polyubiquitinated substrates (Patent Literature 1).

### CITATION LIST

### Patent Literature

Patent Literature 1: Japanese Patent No. 3614866

### Non Patent Literature

Non Patent Literature 1: D. Komander, M. Rape, The ubiquitin code. Annual review of biochemistry 81, 203-229 (2012).

Non Patent Literature 2: Y. T. Kwon, A. Ciechanover, The Ubiquitin Code in the Ubiquitin-Proteasome System and Autophagy. Trends in biochemical sciences 42, 873-886 (2017).

Non Patent Literature 3: R. Yau, M. Rape, The increasing complexity of the ubiquitin code. Nature cell biology 18, 579-586 (2016).

Non Patent Literature 4: C. H. Emmerich, P. Cohen, Optimising methods for the preservation, capture and identification of ubiquitin chains and ubiquitylated proteins by immunoblotting. Biochemical and biophysical research communications 466, 1-14 (2015).

Non Patent Literature 5: R. Hjerpe et al., Efficient protection and isolation of ubiquitylated proteins using tandem ubiquitin-binding entities. EMBO reports 10, 1250-1258 (2009).

Non Patent Literature 6: M. Mattern, J. Sutherland, K. Kadimisetty, R. Barrio, M. S. Rodriguez, Using Ubiquitin Binders to Decipher the Ubiquitin Code. Trends in biochemical sciences 44, 599-615 (2019).

### SUMMARY OF INVENTION

### Technical Problem

The polypeptide monobody described in Patent Literature 1 cannot be said to have high affinity, as with TUBE, and thus, it has been needed to develop a further excellent monobody.

### Solution to Problem

The present inventor has conducted intensive studies directed towards achieving the aforementioned object, and as a result, the present inventor has succeeded in obtaining a synthetic anti-ubiquitin antibody exhibiting excellent effects, thereby completing the present invention.

Specifically, the present invention is as follows.
[1] A synthetic anti-ubiquitin antibody, comprising a fibronectin type III polypeptide, wherein the synthetic antibody comprises a polypeptide of the following (A), (B) or (C):
(A) a polypeptide comprising an FG loop containing a binding region to a ubiquitin, in which the amino acid sequence of the binding region is represented by the following formula (1):

-Ar-Z-Ar-X-Ar- (1)

wherein Ar represents an aromatic amino acid residue, Z represents a single bond or any given amino acid residue, and X represents any given amino acid residue,
(B) a polypeptide consisting of an amino acid sequence comprising a deletion, substitution or addition of one or several amino acids with respect to the amino acid sequence of the polypeptide of the above (A) (except for the amino acid sequence represented by the formula (1)), and having binding activity to the ubiquitin, or
(C) a polypeptide having an identity of at least 80% to the amino acid sequence of the polypeptide of the above (A) (except for the amino acid sequence represented by the formula (1)), and having binding activity to the ubiquitin.
[1-2] The synthetic antibody according to the above [1], wherein Z represents a single bond or any given amino acid residue other than proline and arginine.
[2] The synthetic antibody according to the above [1] or [1-2], wherein the amino acid sequence represented by the formula (1) is FWGW (SEQ ID No: 4), YVYGW (SEQ ID No: 5), YWGW (SEQ ID No: 6), YWWYY (SEQ ID No: 7) or YWMW (SEQ ID No: 8).
[3] The synthetic antibody according to the above [1], [1-2] or [2], wherein the amino acid sequence of the FG loop is YYGNAFWGWP (SEQ ID No: 16), GHTGGSYVYGWY (SEQ ID No: 17), WYAYSYWGWP (SEQ ID No: 18), EYSYMYWGWP (SEQ ID No: 20), WEISYWWYYQ (SEQ ID No: 21) or YYSGYYWMWY (SEQ ID No: 22).
[4] The synthetic antibody according to any one of the above [1] to [3], comprising a polypeptide of the following (P1), (P2) or (P3):
(P1) a polypeptide consisting of the amino acid sequence as set forth in any of SEQ ID Nos: 24, 26, 28, 30, 32 and 34,
(P2) a polypeptide consisting of an amino acid sequence comprising a deletion, substitution or addition of one or several amino acids with respect to the amino acid sequence as set forth in any of SEQ ID Nos: 24, 26, 28, 30, 32 and 34 (except for the amino acid sequence of the binding region to the ubiquitin that corresponds to the amino acid sequence represented by the formula (1)), and having binding activity to the ubiquitin, or
(P3) a polypeptide having an identity of at least 80% to the amino acid sequence as set forth in any of SEQ ID Nos: 24, 26, 28, 30, 32 and 34 (except for the amino acid sequence of the binding region to the ubiquitin that corresponds to the amino acid sequence represented by the formula (1)), and having binding activity to the ubiquitin.
[5] The synthetic antibody according to any one of the above [1] to [4], which has a dissociation constant of 5 nM or less to the ubiquitin.
[6] The synthetic antibody according to any one of the above [1] to [5], wherein the ubiquitin is a polyubiquitin.
[7] DNA encoding a polypeptide of the following (A), (B) or (C):
(A) a polypeptide comprising an FG loop containing a binding region to a ubiquitin, in which the amino acid sequence of the binding region is represented by the following formula (1):

-Ar-Z-Ar-X-Ar- (1)

wherein Ar represents an aromatic amino acid residue, Z represents a single bond or any given amino acid residue, and X represents any given amino acid residue,
(B) a polypeptide consisting of an amino acid sequence comprising a deletion, substitution or addition of one or several amino acids with respect to the amino acid sequence of the polypeptide of the above (A) (except for the amino acid sequence represented by the formula (1)), and having binding activity to the ubiquitin, or
(C) a polypeptide having an identity of at least 80% to the amino acid sequence of the polypeptide of the above (A) (except for the amino acid sequence represented by the formula (1)), and having binding activity to the ubiquitin.
[7-2] The DNA according to the above [7], wherein Z represents a single bond or any given amino acid residue other than proline and arginine.
[8] The DNA according to the above [7] or [7-2], wherein the amino acid sequence represented by the formula (1) is FWGW (SEQ ID No: 4), YVYGW (SEQ ID No: 5), YWGW (SEQ ID No: 6), YWWYY (SEQ ID No: 7) or YWMW (SEQ ID No: 8).
[9] The DNA according to the above [7], [7-2] or [8], wherein the amino acid sequence of the FG loop is YYGNAFWGWP (SEQ ID No: 16), GHTGGSYVYGWY (SEQ ID No: 17), WYAYSYWGWP (SEQ ID No: 18), EYSYMYWGWP (SEQ ID No: 20), WEISYWWYYQ (SEQ ID No: 21) or YYSGYYWMWY (SEQ ID No: 22).
[10] The DNA according to any one of the above [7] to [9], encoding a polypeptide of the following (P1), (P2) or (P3):
(P1) a polypeptide consisting of the amino acid sequence as set forth in any of SEQ ID Nos: 24, 26, 28, 30, 32 and 34,
(P2) a polypeptide consisting of an amino acid sequence comprising a deletion, substitution or addition of one or several amino acids with respect to the amino acid sequence as set forth in any of SEQ ID Nos: 24, 26, 28, 30, 32 and 34 (except for the amino acid sequence of the binding region to the ubiquitin that corresponds to the amino acid sequence represented by the formula (1)), and having binding activity to the ubiquitin, or
(P3) a polypeptide having an identity of at least 80% to the amino acid sequence as set forth in any of SEQ ID Nos: 24, 26, 28, 30, 32 and 34 (except for the amino acid sequence of the binding region to the ubiquitin that corresponds to the amino acid sequence represented by the formula (1)), and having binding activity to the ubiquitin.
[11] The DNA according to any one of the above [7] to [10], which is shown in the following (Q1), (Q2) or (Q3):
(Q1) DNA consisting of the nucleotide sequence as set forth in any of SEQ ID Nos: 23, 25, 27, 29, 31 and 33,
(Q2) DNA hybridizing under stringent conditions with a nucleotide sequence complementary to the nucleotide sequence as set forth in any of SEQ ID Nos: 23, 25, 27, 29, 31 and 33, and having binding activity to the ubiquitin (provided that, among the DNAs hybridizing under the stringent conditions, the nucleotide sequence encoding the amino acid sequence of the binding region to the ubiquitin that corresponds to the amino acid sequence represented by the formula (1) always hybridizes as a codon of the amino acid sequence represented by the formula (1)), or
(Q3) DNA having an identity of at least 80% to the nucleotide sequence as set forth in any of SEQ ID Nos: 23, 25, 27, 29, 31 and 33, and having binding activity to the ubiquitin (provided that, among the DNAs having the identity, the nucleotide sequence encoding the amino acid sequence of the binding region to the ubiquitin that corresponds to the amino acid sequence represented by the formula (1) always encodes the amino acid sequence represented by the formula (1)).
[12] An expression vector, comprising the DNA according to any one of the above [7] to [11].
[13] A transformant, comprising the expression vector according to the above [12].
[14] A method for producing a synthetic antibody, which is characterized in that it

comprises culturing the transformant according to the above [13], and collecting a synthetic anti-ubiquitin antibody from the obtained culture.
[15] A reagent for detecting a ubiquitin or a ubiquitinated substrate, comprising the synthetic antibody according to any one of the above [1] to [6].
[16] A method for detecting a ubiquitin or a ubiquitinated substrate, which is characterized in that it comprises allowing the synthetic antibody according to any one of the above [1] to [6] to come into contact with a test sample.
[17] A method for producing a ubiquitin or a ubiquitinated substrate, which is characterized in that it comprises allowing the synthetic antibody according to any one of the above [1] to [6] to come into contact with a test sample, and collecting a ubiquitin or a ubiquitinated substrate from a complex of the synthetic antibody and the test sample.
[18] The method according to the above [16], wherein the ubiquitin is a polyubiquitin.
[19] The method according to the above [17], wherein the ubiquitin is a polyubiquitin.

### Advantageous Effects of Invention

Since the synthetic antibody of the present invention that is a ubiquitin monobody is superior to existing IgG-type anti-ubiquitin antibodies, can be produced in a large amount in E. coli, and can be easily chemically modified with fluorescent labeling, etc., the present synthetic antibody is expected to be an excellent tool for purification and detection of ubiquitinated substrates in the future.

### BRIEF DESCRIPTION OF DRAWINGS

FIG. 1 is a view showing a ubiquitin modification system and a ubiquitin code. Ubiquitin (Ub) is a proteinaceous post-translational modification molecule with a molecular weight of 8.6 kDa, and is added to a substrate by cascade catalyzed by a series of enzyme group that is composed of a ubiquitin-activating enzyme (E1), a ubiquitin-conjugating enzyme (E2) and a ubiquitin ligase (E3) (ubiquitination). In many cases, ubiquitin is added to ubiquitin itself, thereby forming a polyubiquitin chain. In a cell, there are 8 types of polyubiquitin chains having different structures (K6, K11, K27, K33, K48 and K63 chains that are linked via different lysine residues (K), and M1 chain linked via an N-terminal methionine residue (M)). Among them, a K48-linked ubiquitin chain, which is linked via the 48th lysine residue, is used as a signal for degradation of a substrate protein by proteasome. On the other hand, a K63-linked ubiquitin chain regulates membrane protein endocytosis, signal transduction, DNA repair, etc. Other ubiquitin chains have also been suggested to have specific functions, and further recently, mixed chains linked by a different linkage formula, branched chains, and further, ubiquitins have been found to be phosphorylated or acetylated. Therefore, the functional information contained in a wide variety of ubiquitin modifications is called ubiquitin code. The ubiquitin-binding protein having a ubiquitin-binding domain (UBD) is important for the functional expression of ubiquitin codes, and UBD is also called a decoder molecule because it identifies the higher-order structure of a ubiquitin chain and transmits the information to downstream molecules. Moreover, since ubiquitin chains are cleaved by deubiquitinase (DUB), ubiquitin modification is a reversible post-translational modification.

FIG. 2 is a comparison view of IgG and a synthetic antibody (Monobody). FIG. 2A shows the structure of a human immunoglobulin (IgG) antibody. The antibody has a structure in which two heavy chains and two light chains are connected by disulfide bonds (S-S). Both chains consist of constant domains (CL and CH1-CH3) and variable domains (VL and VH). The variable domain contains complementarity-determining regions (CDRs) that determine the substrate-binding specificity of the antibody. The conventional anti-ubiquitin monoclonal antibody (P4D1) and anti-ubiquitin chain antibody (FK2) are both mouse IgGs. FIG. 2B shows the structure of a synthetic antibody (monobody). The monobody is a synthetic antibody comprising a human fibronectin type 3 domain as a backbone. Like the IgG constant domain, the monobody has 2 CDR-like loops from a barrel-shaped scaffold consisting of 7 β-sheets. In the present invention, a ubiquitin-specific monobody was obtained using a synthetic antibody library in which the amino acid sequences of the loops are randomized. FIG. 2C shows the DNA sequence of the monobody. The bolded regions correspond to the variable loops, and the sequence of wild-type human fibronectin is shown herein. The nucleotide sequence encoding the wild-type human fibronectin is shown in SEQ ID No. 37, and the amino acid sequence of the wild-type human fibronectin is shown in SEQ ID No. 38. In the random library, the lengths of the variable loops are also varied, and loop 1 consists of 8 or 10 amino acids, whereas loop 2 consists of 10 or 12 amino acids.

FIG. 3 shows in vitro selection of monobody against ubiquitin. FIG. 3A shows that monobodies interacting with biotin-labeled ubiquitin monomers were screened from a library (10³ diversity), in which the two loops of monobody were randomized,, according to a TRAP (Transcription-translation coupled with association of puromycin linker) method. FIG. 3B shows that a total of five rounds of ubiquitin-targeted screening were performed, and monobody clones against ubiquitin were concentrated. The DNA sequences of the fifth round of monobody clone mixtures were determined with Next-Generation Sequencer (NGS), and total of 7 clones having different sequences in each of the two loops were obtained. In the right panel of FIG. 3B, loop 1 represents a BC loop and loop 2 represents an FG loop. The amino acid sequences of clones A to G of loop 1 are shown in SEQ ID Nos: 9 to 15, respectively. In addition, the amino acid sequences of clones A to G of loop 2 are shown in SEQ ID Nos: 16 to 22, respectively.

FIG. 4 is a view showing the results obtained by measuring the binding affinity of monobody and ubiquitin. FIG. 4A shows a construct used in the binding experiment. A recombinant protein, in which a monobody, a Nus tag as a tag for solubilization, and a hexahistidine tag (His6) for purification were fused from the N-terminus, was prepared in an E. coli expression system. 7 clones of monobodies shown in FIG. 3B were prepared. FIG. 4B shows determination of dissociation constants by a bio-layer interferometry method (BLI). A biotinylated ubiquitin was immobilized and the interaction was analyzed using the 6 recombinant proteins shown in FIG. 4A as analytes. The dissociation constant (Kd) was about 1 nM for all of the analytes.

FIG. 5 is a view showing the results of Western blot analysis using cell extracts. FIG. 5A shows the biotin labeling of the ubiquitin monobody. GST-His6-Monobody was cleaved to remove the GST-His6 tag by TEV protease, and the generated N-terminal GGGS sequence and the biotin-labeled peptide (Biotin-PEG12-VLLPRTGG) were peptide-ligated using Sortase. The underline indicates the target sequence of Sortase. The final product is Biotin-VLLPRTGGGS-Monobody. FIG. 5B shows that human colon cancer-derived HCT-116 cells were treated with a ubiquitin-activating enzyme E1 inhibitor (TAK-243, 1 uM) or a proteasome inhibitor (Btz, 1 uM) for 3 hours, and that proteins were extracted and were then subjected to Western blot analysis. It has been known that polyubiquitinated substrates are increased and ubiquitinylated histones are decreased by proteasome inhibition, and that polyubiquitinated substrates are decreased and ubiquitin monomers are increased by E1 inhibition. Using a biotin-labeled anti-ubiquitin monobody (Ub monobody) and an existing IgG-type anti-ubiquitin antibody (FK2 or P4D1) as antibodies for detecting ubiquitin or ubiquitinated substrate, band patterns were compared. In addition, a wild-type monobody (control monobody) was used as a negative control. Although the reactivity differed depending on the antibody, polyubiquitinated substrates could be detected by all of them. Moreover, the ubiquitin monomer was detectable only with P4D1. On the other hand, the anti-ubiquitin monobody was highly reactive to ubiquitinylated histone (monoubiquitinylated H2A), although it could not detect ubiquitin monomer.

FIG. 6 is a view showing immunoprecipitation of ubiquitinated proteins from cell extracts. FIG. 6A shows a ubiquitin monobody construct used in immunoprecipitation. A recombinant protein, in which a hexahistidine tag (His6) for purification, a Halo tag for solidification, and a monobody were fused from the N-terminus, was prepared in an E. coli expression system. As monobodies, wild-type monobody (control monobody) and monobody against ubiquitin (Ub monobody, clone C) were prepared. FIG. 6B shows an electrophoretic image of a purified preparation of Halo tag-fused monobody. Purified proteins were separated by SDS-PAGE and were then subjected to CBB staining. FIG. 6C shows the Western blot analysis of immunoprecipitates. Using mouse control IgG (Control IgG), mouse anti-ubiquitin antibodies (FK2 and P4D1), control monobody (Control monobody), and anti-ubiquitin monobody (Ub monobody), ubiquitinated substrate and ubiquitin were immunoprecipitated from the extract of human colon cancer-derived HCT-116 cells. Subsequently, the immunoprecipitates were analyzed by Western blotting with an anti-ubiquitin antibody (P4D1). Input corresponds to 20% of the extract subjected to the immunoprecipitation. The signal intensities of polyubiquitinated substrates were in the order of ubiquitin monobody>FK2>P4D1. In addition, ubiquitinylated histone (monoubiquitinylated H2A) and ubiquitin monomer were detected in the ubiquitin monobody.

FIG. 7 is a view showing the results of quantitative mass spectrometry analysis of immunoprecipitates. FIG. 7A shows absolute quantification of ubiquitin chains. The immunoprecipitates subjected to FIG. 5C were electrophoresed, and high molecular weight regions (62 kDa or more) were subjected to quantitative mass spectrometry analysis. A stable isotope-labeled ubiquitin-derived peptide (name: EST) as an internal standard and 8 types of ubiquitin chain-derived peptides (names: K6, K11, K27, K29, K33, K48, K63, and M1) were used, and the quantification values (fmol) were described. Since the EST peptide sequence overlaps with the K63 chain peptide sequence, the total ubiquitin value was calculated by adding the value of the K63 chain-derived peptide to the quantitative value of the EST peptide. The total amount of ubiquitin contained in the ubiquitin monobody immunoprecipitates was confirmed to be higher than that of FK2 or P4D1. FIG. 7B shows the composition of the 8 types of ubiquitin chains. The value of the control is subtracted from the value of each antibody, a total value of the 8 types of ubiquitin chain-derived peptides is set at 100%, and the percentage of each ubiquitin chain is shown as a pie chart. Since the percentage of each ubiquitin chain in the cell extract (Input) and the percentage of each ubiquitin chain in the FK2 and ubiquitin monobody immunoprecipitates are almost identical to each other, it was suggested that FK2 and ubiquitin monobody can bind to the major ubiquitin chains without bias.

FIG. 8 is a view showing the results of immunostaining. FIG. 8A shows the fluorescent labeling of ubiquitin monobody. The GST-monobody shown in FIG. 5A was cleaved to remove the GST-His6 tag by TEV protease, and the generated N-terminal GGGS sequence and the TAMRA peptide (TAMRA-PEG12-VLLPRTGG) were peptide-ligated using Sortase. The underline indicates the target sequence of Sortase. The final product is TAMRA-VLLPRTGGGS-Monobody. FIG. 8B shows electrophoretic images of TAMRA-labeled ubiquitin monobody. The left view shows a CBB-stained image, and the right view shows a fluorescence detection image. FIG. 8C shows the staining of ubiquitin monobody using cells stably expressing puromycin-treated EGFP-ubiquitin. Human colon cancer-derived HCT116 cells stably expressing EGFP-ubiquitin were used. EGFP-ubiquitin signals are mainly detected in the nucleoplasm and cytoplasm, but a puromycin treatment causes formation of ubiquitin-positive foci by a newborn defective protein in the cytoplasm (left panel). When cell staining was carried out using TAMRA-labeled ubiquitin monobody (1 ug/ml and 0.1 ug/ml), the result were almost completely matched with the fluorescent signals of EGFP-ubiquitin. FIG. 8D shows the fluorescence-stained images of puromycin-treated EGFP-ubiquitin stably expressing cells using the anti-ubiquitin antibody FK2. Fluorescence images were obtained using a commonly used IgG-type anti-ubiquitin antibody FK2 (10 ug/ml) as a primary antibody and an Alexa568-labeled anti-mouse IgG antibody as a secondary antibody under the same conditions as FIG. 8C, including cells and exposure time. As a result, the FK2 antibody was detected in cytoplasmic foci, but intranuclear ubiquitin was not detected.

FIG. 9 is a view showing a comparison made among individual ubiquitin antibodies. With regard to Western blotting (WB) and immunoprecipitation/pull-down, ubiquitin monomer, monoubiquitinylated histone, and polyubiquitin chain (including polyubiquitinated substrate) were entered separately. The reactivity intensity is indicated by the number of “+”. TUBE is not applicable for West blot analysis (N.A.; not applicable).

FIG. 10 is a view showing the structure of a complex of a synthetic antibody (Monobody) and a ubiquitin (Ub). The view shows a ribbon model of the X-ray crystal structure of a synthetic antibody (monobody) recognizing a ubiquitin. A complex was formed with a ubiquitin at the loop 2 side of the complementarity-determining region of the variable domain. Loop 1 and loop 2, and the side chain of the ubiquitin recognized by the Monobody are shown as sticks.

FIG. 11 is a view showing the responsible residues for the interaction between the synthetic antibody (Monobody) and the ubiquitin (Ub). Regarding loop 2, I44 and V70 of ubiquitin hydrophobically interacted with W91 of Monobody, and W89 of ubiquitin with R42 of Monobody, respectively. In addition, R72 of ubiquitin bound with S87 of Monobody and Q49 of ubiquitin with Y84 of Monobody via hydrogen bonds, respectively. Regarding loop 1, R42 of ubiquitin bound with D33 of Monobody via a hydrogen bond. Hydrogen bonds were observed from V70 to I73 on the C-terminal side of ubiquitin, and in the main chain from W88 to Y91 of loop 2 of Monobody.

FIG. 12 is a view showing the results of a polyubiquitination inhibition experiment. a) K48 chain, b) K63 chain.

### DESCRIPTION OF EMBODIMENTS

The present invention relates to a synthetic anti-ubiquitin antibody comprising a fibronectin type III polypeptide. In the present invention, a polypeptide specifically binding to a ubiquitin is also referred to as a “ubiquitin monobody,” or is simply referred to as a “monobody.” Since the monobody is a synthetic antibody specifically binding to a ubiquitin, the term “ubiquitin monobody” or “monobody” has the same definitions as those of the “synthetic antibody” in the present description.

The synthetic antibody of the present invention comprises a polypeptide of the following (A), (B) or (C):
(A) a polypeptide comprising an FG loop containing a binding region to a ubiquitin, in which the amino acid sequence of the binding region is represented by the following formula (1):

-Ar-Z-Ar-X-Ar- (1)

wherein Ar represents an aromatic amino acid residue, Z represents a single bond or any given amino acid residue, and X represents any given amino acid residue,
(B) a polypeptide consisting of an amino acid sequence comprising a deletion, substitution or addition of one or several amino acids with respect to the amino acid sequence of the polypeptide monobody of the above (A) (except for the amino acid sequence represented by the formula (1)), and having binding activity to the ubiquitin, or
(C) a polypeptide having an identity of at least 80% to the amino acid sequence of the polypeptide monobody of the above (A) (except for the amino acid sequence represented by the formula (1)), and having binding activity to the ubiquitin.

Herein, Z is preferably a single bond, or any given amino acid residue other than proline and arginine.

In one aspect of the present invention, the amino acid sequence represented by the formula (1), namely, the amino acid sequence of the binding region to the ubiquitin in the FG loop can be expressed with, for example, WGW (SEQ ID No: 4), YVYGW (SEQ ID No: 5), YWGW (SEQ ID No: 6), YWWYY (SEQ ID No: 7) or YWMW (SEQ ID No: 8). The amino acid sequence of the FG loop comprising these amino acid sequences can be expressed with, for example, YYGNAFWGWP (SEQ ID No: 16), GHTGGSYVYGWY (SEQ ID No: 17), WYAYSYWGWP (SEQ ID No: 18), EYSYMYWGWP (SEQ ID No: 20), WEISYWWYYQ (SEQ ID No: 21) or YYSGYYWMWY (SEQ ID No: 22).

### 1. Outline

Ubiquitin modification is a post-translational modification essential for living organisms, and regulates various cellular functions such as signaling, DNA repair, and transport of membrane proteins, in addition to protein quality control. To date, anti-ubiquitin antibodies and artificial proteins such as TUBE have been developed to detect ubiquitin modification (ubiquitination) of proteins. However, they have problems such as low affinity to ubiquitin monomers and large size as probes.

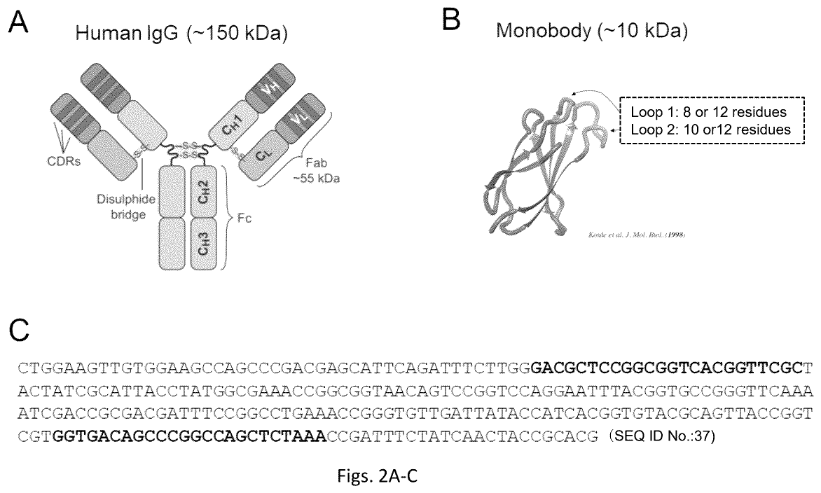

Thus, in the present invention, it was attempted to obtain a monobody specific to ubiquitin, using a random library of small-molecule-type synthetic antibody monobodies (human fibronectin skeleton, about 9.4 kDa), and as a result, clone monobodies with different sequences were obtained. The monobody is a synthetic antibody comprising a human fibronectin type 3 (FN3) domain as a skeleton, and has two variable loops serving as complementarity-determining regions from a barrel-shaped scaffold consisting of seven β-sheets (FIG. 2).

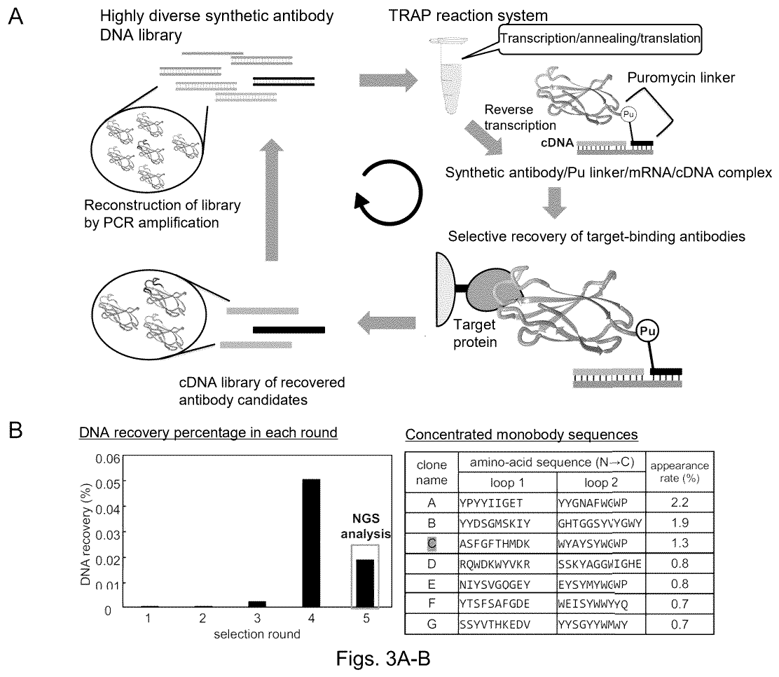

In the present invention, a ubiquitin-specific monobody was obtained using a synthetic antibody library, in which the amino acid sequences of these two loops are randomized, according to a TRAP (transcription-translation coupled with association of puromycin linker) presentation method (T. Kondo et al., Antibody-like proteins that capture and neutralize SARS-CoV-2. Sci Adv 6 (2020).) (FIG. 3). In FIG. 3, FIG. 3A is a schematic view of the TRAP presentation method. Subsequently, the anti-ubiquitin monobody was compared with an IgG-type anti-ubiquitin antibody, in terms of immunoprecipitation ability and immunostaining ability.

As a result, it was confirmed: that the interaction between the ubiquitin monobody and the ubiquitin monomer has a high affinity with a dissociation constant of several nM, and the anti-ubiquitin monobody is able to immunoprecipitate a ubiquitinated substrate more efficiently than the conventional IgG-type anti-ubiquitin antibody; and that the anti-ubiquitin monobody is able to capture various linkage modes of ubiquitin chains. The ubiquitin monobody can also be applied to immunostaining, and could detect ubiquitinated substrates in cells with higher sensitivity and higher accuracy than the conventional IgG-type anti-ubiquitin antibody. Thus, the present ubiquitin monobody is superior to existing IgG-type anti-ubiquitin antibodies, can be produced in a large amount in E. coli, and can be used as a synthetic anti-ubiquitin antibody because of the ease of chemical modification such as fluorescent labeling, etc., and thus, the present ubiquitin monobody is expected to be an excellent tool for the purification and detection of ubiquitinated substrates.

### 2. Screening of Ubiquitin Monobody

In the present invention, various screening methods can be utilized used to obtain a ubiquitin monobody from a library.

### (1) Preparation of Biotinylated Ubiquitin

First, a construct was prepared by adding a Cys residue for labeling and a tag for purification to the C-terminal region of a human-derived ubiquitin, and it is then purified by an E. coli expression system. Then, a label is added to the Cys residue.

The E. coli expression system is a known system and can be carried out by utilizing a chemical reaction selective for Cys residues to expressed proteins. In addition, the labeling is not particularly limited, and examples of the labeling substance may include biotin and desthiobiotin.

### (2) TRAP Presentation Method

The library used in the present invention is, for example, a small-molecule-type synthetic antibody monobody FN3 (fibronectin type III) mRNA library (equivalent to 10¹³). The library can be obtained by performing in vitro transcription using RNA polymerase, after a DNA library is prepared by linking chemically synthesized DNAs. From this library, FN3 protein is translated in vitro, and clones that interact with ubiquitin are selected using a biotinylated ubiquitin as a bait. Protein expression in test tubes (in vitro protein expression) is a technique of producing a recombinant protein in a lysate or in E. coli by utilizing a biomolecular translation mechanism extracted from cells. Since protein synthesis occurs in a cell lysate or in E. coli, rather than in cultured cells, this technique is also called cell-free protein expression.

The translation technique used in one aspect of the present invention is a known cell-free translation technique, for example, a “reconstituted cell-free translation system” (PURE system). In the present invention, a technique called a reconstituted system is adopted, in which factors necessary for translation (ribosomes and other proteins) are expressed and purified in E. coli, and are then mixed with one another.

This screening process is repeated multiple times to concentrate clones that bind to ubiquitin with high affinity. Subsequently, the nucleotide sequence of a variable region is determined using Next-Generation DNA Sequencer to obtain anti-ubiquitin monobody clones.

### 3. Measurement of Binding Affinity between Monobody and Ubiquitin

### (1) Expression and Purification of Monobody Protein in E. coli

A construct harboring a monobody, a Nus tag for solubilization, and a hexahistidine tag (His6) for purification from the N-terminus was prepared. Subsequently, a recombinant protein was expressed in E. coli and purified using the His6 tag.

### (2) Determination of Dissociation Constant by Bio-Layer Interferometry Method (BLI)

A biotinylated ubiquitin (5 nM) was immobilized on a streptavidin sensor, and binding and dissociation were then monitored using an anti-ubiquitin monobody (2-fold dilution series of 0.625 to 10 nM) as an analyte. Curve fitting was carried out using the specialized software ForteBio Data Analysis ver 10.0, and the dissociation constant (Kd) was calculated from the calculated first-order reaction approximation curve.

### 4. Monobody

### (1) Monobody Polypeptide and DNA

The monobody obtained by the above-described screening and binding affinity measurement is a synthetic antibody comprising a fibronectin III (FN3) type polypeptide as a skeleton. The FN3 type monobody contains a BC loop, a CD loop, a DE loop, and an FG loop. In the present invention, the loop involved in the affinity with ubiquitin is the FG loop.

In the present invention, the amino acid sequences of the BC loop and the FG loop were analyzed, and as a result, it was found that there is a common motif in the FG loop as an amino acid sequence in the region involved in the binding to ubiquitin. The amino acid sequence can be shown in the following formula (1):

-Ar-Z-Ar-X-Ar- (1)

### (SEQ ID No: 1).

In the above formula (1), Ar represents an aromatic amino acid residue, Z represents a sinble bond or any given amino acid residue, and X represents any given amino acid residue. The term “any given amino acid residue” means 20 types amino acid residues that are structural components of a protein, namely, means acidic amino acids having two carboxyl groups in the structure thereof (aspartic acid (Asp or D) or glutamic acid (Glu or E)), basic amino acids having two or more amino groups (lysine (Lys or K), arginine (Arg or R) or histidine (His or H)), and neutral amino acids. Neutral amino acids are classified as follows, according to the groups of the side chains constituting the amino acids. That is, neutral amino acids are classified into (i) glycine (Gly or G), alanine (Ala or A), valine (Val or V), leucine (Leu or L) or isoleucine (Ile or I) having alkyl chains, (ii) serine (Ser or S) or threonine (Thr or T) having a hydroxy group, (iii) cysteine (Cys or C) or methionine (Met or M) containing sulfur, (iv) asparagine (Asn or N) or glutamine (Gln or Q) having an amide group, (v) proline (Pro or P) having an imino group, (vi) phenylalanine (Phe or F), tyrosine (Tyr or Y) or tryptophan (Trp or W) having an aromatic group.

When Z is a single bond, the formula (1) becomes the amino acid sequence shown in the following formula (2):

-Ar-Ar-X-Ar- (2)

(SEQ ID No: 2), and when Z represents any given amino acid residue, the formula (1) becomes the amino acid sequence shown in the following formula (3):

-Ar-X-Ar-X-Ar- (3)

### (SEQ ID No: 3).

However, in the present invention, when Z represents an amino acid residue, it is preferably any given amino acid residue other than proline and arginine.

Herein, the “amino acid sequence in the region involved in the binding” comprises at least two amino acid residues of the amino acid sequence shown in the formula selected from the above formulae (1) to (3).

In the formulae (1) to (3), Ar represents an aromatic amino acid residue, which is phenylalanine (Phe or F), tyrosine (Tyr or Y) or tryptophan (Trp or W).

The amino acid sequence shown in the above formula (2) is, for example, FWGW (SEQ ID No: 4), YWGW (SEQ ID No: 6), or YWMW (SEQ ID No: 8). The amino acid sequence shown in the above formula (3) is, for example, YVYGW (SEQ ID No: 5) or YWWYY (SEQ ID No: 7).

Moreover, in one aspect of the present invention, the amino acid sequence of the FG loop is YYGNAFWGWP (SEQ ID No: 16), GHTGGSYVYGWY (SEQ ID No: 17), WYAYSYWGWP (SEQ ID No: 18), EYSYMYWGWP (SEQ ID No: 20), WEISYWWYYQ (SEQ ID No: 21) or YYSGYYWMWY (SEQ ID No: 22).

Accordingly, the monobody of the present invention comprises
(A) a polypeptide comprising an FG loop containing a binding region to a ubiquitin, in which the amino acid sequence of the binding region is represented by the following formula (1):

-Ar-Z-Ar-X-Ar- (1)

wherein Ar represents an aromatic amino acid residue, Z represents a single bond or any given amino acid residue, and X represents any given amino acid residue.

In one aspect of the present invention, Z represents a single bond, or any given amino acid residue other than proline and arginine.

Moreover, the monobody of the present invention comprises a polypeptide of the following (B) or (C). It is to be noted that the polypeptide of the following (B) or (B), or DNA encoding the polypeptide, is a mutant of the polypeptide (A), and the polypeptide of the aspect of (B) or (C) may be simply referred to as a “mutant” or a “mutant monobody” at times in the present description.
(B) A polypeptide consisting of an amino acid sequence comprising a deletion, substitution or addition of one or several amino acids with respect to the amino acid sequence of the polypeptide of the above (A), and having binding activity to the ubiquitin.
(C) A polypeptide having an identity of at least 80% to the amino acid sequence of the polypeptide of the above (A), and having binding activity to the ubiquitin.

However, even in a case where the amino acid sequence comprises a deletion, substitution or addition of one or several amino acids, the mutation of the aspect having binding activity to the ubiquitin, or the mutation of the aspect having an identity of at least 80% and having binding activity to the ubiquitin includes, for example, a mutation in the site other than the position of amino acids essential for the binding in the sequence within the loop. Accordingly, with regard to the amino acid sequence represented by the formula (1) within the FG loop, the amino acid sequence comprising a deletion, substitution or addition of one or several amino acids is excluded in the polypeptide of the above (B), and the amino acid sequence having an identity of at least 80% is excluded in the polypeptide of the above (C).

Since the regions other than the FG loop (the fibronectin skeleton and other loops) are not necessarily essential for the binding to ubiquitin, the sequences in these regions may comprise the mutations of the above-described aspects.

In one aspect of the monobody of the present invention, polypeptides comprising the FG loop are as follows:
(P1) a polypeptide consisting of the amino acid sequence as set forth in any of SEQ ID Nos: 24, 26, 28, 30, 32 and 34,
(P2) a polypeptide consisting of an amino acid sequence comprising a deletion, substitution or addition of one or several amino acids with respect to the amino acid sequence as set forth in any of SEQ ID Nos: 24, 26, 28, 30, 32 and 34 (except for the amino acid sequence of the binding region to the ubiquitin that corresponds to the amino acid sequence represented by the formula (1)), and having binding activity to the ubiquitin, and
(P3) a polypeptide having an identity of at least 80% to the amino acid sequence as set forth in any of SEQ ID Nos: 24, 26, 28, 30, 32 and 34 (except for the amino acid sequence of the binding region to the ubiquitin that corresponds to the amino acid sequence represented by the formula (1)), and having binding activity to the ubiquitin.

Herein, the above-described “amino acid sequence comprising a deletion, substitution or addition of one or several amino acids” may be an amino acid sequence comprising a deletion, substitution or addition of, for example, 1 to 10 amino acids, 1 to 9 amino acids, 1 to 8 amino acids, 1 to 7 amino acids, 1 to 6 amino acids, 1 to 5 amino acids, 1 to 4 amino acids, 1 to 3 amino acids, 1 or 2 amino acids, or 1 amino acid.

When a peptide is prepared by genetic engineering, the above-described introduction of mutations, such as deletion, substitution or addition, can be carried out using a mutagenesis kit that utilizes a site-specific mutagenesis method, for example, using GenEdit Site-Directed Mutagenesis Kit (Funakoshi), PrimeSTAR (registered trademark) Mutagenesis Basal Kit (TaKaRa), etc. In addition, whether it is a peptide into which the above-described deletion, substitution, or addition mutation has been introduced can be confirmed by various types of amino acid sequence determination methods, structure analysis methods such as mass spectrometry, etc.

Furthermore, in the present invention, the binding mode of a mutant (mutant monobody) to a ubiquitin can be confirmed or predicted by the three-dimensional structure analysis of the mutant monobody. Any methods such as computerized three-dimensional structure analysis, crystal structure analysis, etc. can be employed as such three-dimensional structure analysis.

In the present invention, the “binding activity to the ubiquitin” means that the ubiquitin binds to the amino acid residue in the FG loop constituting the monobody via a non-covalent bond, and this activity can be measured by, for example, a dissociation constant determination method involving the above-described biolayer interferometry method, a surface plasmon resonance method, etc.

In the present invention, the affinity of a monobody binding to a ubiquitin can be expressed by the dissociation constant (Kd), which is 5 nM or less, for example, 0.5 to 3 nM, or 0.8 to 2.8 nM.

Herein, the ubiquitin includes not only a polyubiquitin, but also a substrate protein-binding ubiquitin or a polyubiquitin.

Once the monobody of the present invention has been obtained by the above-described screening method and the amino acid sequence and nucleotide sequence of the monobody have been analyzed, the monobody can be produced by genetic engineering. In addition, a mutant polypeptide comprising a deletion, substitution or addition of one or several amino acids in the amino acid sequence of the polypeptide constituting the present monobody (which is also referred to as a “monobody polypeptide”), and having binding activity to a ubiquitin, can be obtained by designing and synthesizing the amino acid sequence of the mutant polypeptide and selecting clones that interact with the ubiquitin in the same manner as that described above.

The monobody of the present invention can be obtained by artificial chemical synthesis. Examples of the synthetic method may include an azide method, an acid chloride method, an acid anhydride method, a mixed acid anhydride method, an active ester method, and an oxidation-reduction method, In addition, both a solid phase synthetic method and a liquid phase synthetic method can be applied for the synthesis. Commercially available peptide synthesizers may also be used. After the synthesis reaction, a monobody of interest can be purified by a combination of known purification methods such as chromatography.

Moreover, in the present invention, the DNA encoding the monobody polypeptide may be incorporated into various types of expression vectors, etc., and may be introduced into cells, so that it is allowed to express therein, according to known genetic recombination technology (e.g., Sambrook J. et al., Molecular Cloning, A Laboratory Manual (4th edition), Cold Spring Harbor Laboratory Press (2012)), and then, the monobody polypeptide may be obtained by known recovery and purification methods.

In one aspect of the present invention, the DNA encoding the monobody polypeptide may be DNA encoding the polypeptide of the following (A), (B) or (C): (A) a polypeptide comprising an FG loop containing a binding region to a ubiquitin, in which the amino acid sequence of the binding region is represented by the following formula (1):

-Ar-Z-Ar-X-Ar- (1)

wherein Ar represents an aromatic amino acid residue, Z represents a single bond or any given amino acid residue, and X represents any given amino acid residue,
(B) a polypeptide consisting of an amino acid sequence comprising a deletion, substitution or addition of one or several amino acids with respect to the amino acid sequence of the polypeptide of the above (A) (except for the amino acid sequence represented by the formula (1)), and having binding activity to the ubiquitin, or
(C) a polypeptide having an identity of at least 80% to the amino acid sequence of the polypeptide of the above (A) (except for the amino acid sequence represented by the formula (1)), and having binding activity to the ubiquitin.

In the above (A), Z is preferably a single bond, or any given amino acid residue other than proline and arginine.

Furthermore, in one aspect of the present invention, the DNA encoding the monobody polypeptide may be DNA encoding the polypeptide of the following (P1), (P2) or (P3):
(P1) a polypeptide consisting of the amino acid sequence as set forth in any of SEQ ID Nos: 24, 26, 28, 30, 32 and 34,
(P2) a polypeptide consisting of an amino acid sequence comprising a deletion, substitution or addition of one or several amino acids with respect to the amino acid sequence as set forth in any of SEQ ID Nos: 24, 26, 28, 30, 32 and 34 (except for the amino acid sequence of the binding region to the ubiquitin that corresponds to the amino acid sequence represented by the formula (1)), and having binding activity to the ubiquitin, or
(P3) a polypeptide having an identity of at least 80% to the amino acid sequence as set forth in any of SEQ ID Nos: 24, 26, 28, 30, 32 and 34 (except for the amino acid sequence of the binding region to the ubiquitin that corresponds to the amino acid sequence represented by the formula (1)), and having binding activity to the ubiquitin.

In one aspect of the present invention, a specific example of such DNA may be DNA of the following (Q1), (Q2) or (Q3):
(Q1) DNA consisting of the nucleotide sequence as set forth in any of SEQ ID Nos: 23, 25, 27, 29, 31 and 33,
(Q2) DNA hybridizing under stringent conditions with a nucleotide sequence complementary to the nucleotide sequence as set forth in any of SEQ ID Nos: 23, 25, 27, 29, 31 and 33, and having binding activity to the ubiquitin, and
(Q3) DNA having an identity of at least 80% to the nucleotide sequence as set forth in any of SEQ ID Nos: 23, 25, 27, 29, 31 and 33, and having binding activity to the ubiquitin.

As described above, since the regions other than the FG loop (the fibronectin skeleton and other loops) are not necessarily essential for the binding to ubiquitin, the sequences in these regions may comprise the mutations of the above-described aspects.

However, in the present invention, among the above-described DNAs that hybridize under stringent conditions, the nucleotide sequence encoding the amino acid sequence of the binding region to the ubiquitin corresponding to the amino acid sequence represented by the formula (1) always hybridizes as a codon of the amino acid sequence represented by the formula (1).

In addition, among the DNAs having an identity, the nucleotide sequence encoding the amino acid sequence of the binding region to the ubiquitin corresponding to the amino acid sequence represented by the formula (1) always encodes the amino acid sequence represented by the formula (1).

The DNAs and amino acid sequences of the above-described ubiquitin-binding monobodies are shown in Table 1.

TABLE 1

| | | | SEQ |
|---|---|---|---|
| | | | ID |
| Clone | Kd | Sequence | No. |
| A | 1.1 | (DNA sequence) | 23 |
| | nM | CTGGAAGTTGTGGAAGCCAGCCCGACGAGCATTCAGATTTCTTGGGACG | |
| | | CTTACCCGTACTACATCATCGGTGAAACTGTTCGCTACTATCGCATTAC | |
| | | CTATGGCGAAACCGGCGGTAACAGTCCGGTCCAGGAATTTACGGTGCCG | |
| | | GGTTCAAAATCGACCGCGACGATTTCCGGCCTGAAACCGGGTGTTGATT | |
| | | 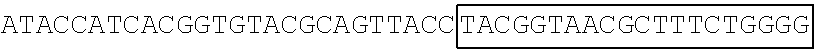 | |
| | | 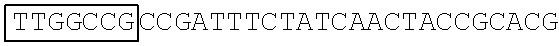 | |
| | | (Amino acid sequence) | 24 |
| | | LEVVEASP-TSIQISWDA-YPYYIIGET-VRYYRITYGETG-GNSPVQE | |
| | | 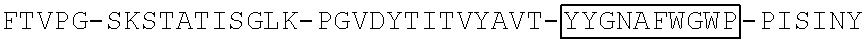 | |
| | | RT | |
| B | 1.5 | (DNA sequence) | 25 |
| | nM | CTGGAAGTTGTGGAAGCCAGCCCGACGAGCATTCAGATTTCTTGGGACG | |
| | | CTTACTACGACTCTGGTATGTCTAAAATCTACGTTCGCTACTATCGCAT | |
| | | TACCTATGGCGAAACCGGCGGTAACAGTCCGGTCCAGGAATTTACGGTG | |
| | | CCGGGTTCAAAATCGACCGCGACGATTTCCGGCCTGAAACCGGGTGTTG | |
| | | 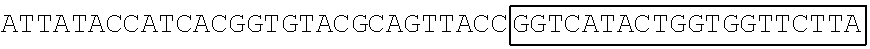 | |
| | | 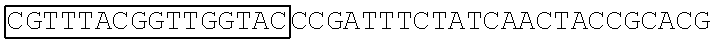 | |
| | | (Amino acid sequence) | 26 |
| | | LEVVEASP-TSIQISWDA-YYDSGMSKIY-VRYYRITYGETG-GNSPVQ | |
| | | 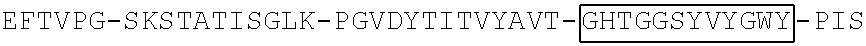 | |
| | | INYRT | |
| C | 0.88 | (DNA sequence) | 27 |
| | nM | CTGGAAGTTGTGGAAGCCAGCCCGACGAGCATTCAGATTTCTTGGGACG | |
| | | CTGCTTCTTTCGGTTTCACTCATATGGACAAAGTTCGCTACTATCGCAT | |
| | | TACCTATGGCGAAACCGGCGGTAACAGTCCGGTCCAGGAATTTACGGTG | |
| | | CCGGGTTCAAAATCGACCGCGACGATTTCCGGCCTGAAACCGGGTGTTG | |
| | | 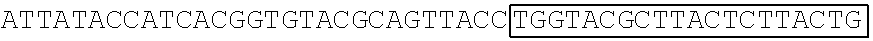 | |
| | | 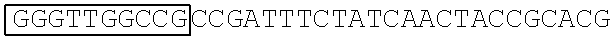 | |
| | | (Amino acid sequence) | 28 |
| | | LEVVEASP-TSIQISWDA-ASFGFTHMDK-VRYYRITYGETG-GNSPVQ | |
| | | 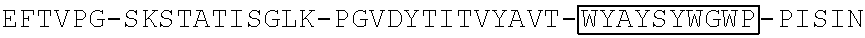 | |
| | | YRT | |
| E | 1.2 | (DNA sequence) | 29 |
| | nM | CTGGAAGTTGTGGAAGCCAGCCCGACGAGCATTCAGATTTCTTGGGACG | |
| | | CTAACATCTACTCTGTTGGTCAGGGTGAATACGTTCGCTACTATCGCAT | |
| | | TACCTATGGCGAAACCGGCGGTAACAGTCCGGTCCAGGAATTTACGGTG | |
| | | CCGGGTTCAAAATCGACCGCGACGATTTCCGGCCTGAAACCGGGTGTTG | |
| | | 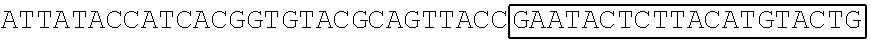 | |
| | | 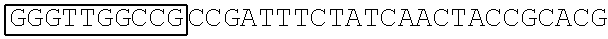 | |
| | | (Amino acid sequence) | 30 |
| | | LEVVEASP-TSIQISWDA-NIYSVGQGEY-VRYYRITYGETG-GNSPVQ | |
| | | 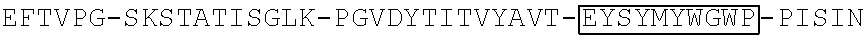 | |
| | | YRT | |
| F | 2.8 | (DNA sequence) | 31 |
| | nM | CTGGAAGTTGTGGAAGCCAGCCCGACGAGCATTCAGATTTCTTGGGACG | |
| | | CTTACACTTCTTTCTCTGCTTTCGGTGACGAAGTTCGCTACTATCGCAT | |
| | | TACCTATGGCGAAACCGGCGGTAACAGTCCGGTCCAGGAATTTACGGTG | |
| | | CCGGGTTCAAAATCGACCGCGACGATTTCCGGCCTGAAACCGGGTGTTG | |
| | | 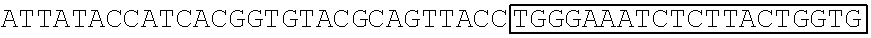 | |
| | | 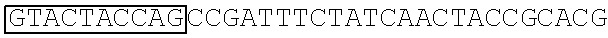 | |
| | | (Amino acid sequence) | 32 |
| | | LEVVEASP-TSIQISWDA-YTSFSAFGDE-VRYYRITYGETG-GNSPVQ | |
| | | 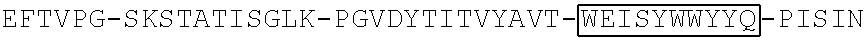 | |
| | | YRT | |
| G | 1.2 | (DNA sequence) | 33 |
| | nM | CTGGAAGTTGTGGAAGCCAGCCCGACGAGCATTCAGATTTCTTGGGACG | |
| | | CTTCTTCTTACGTTACTCATAAAGAAGACGTTGTTCGCTACTATCGCAT | |
| | | TACCTATGGCGAAACCGGCGGTAACAGTCCGGTCCAGGAATTTACGGTG | |
| | | CCGGGTTCAAAATCGACCGCGACGATTTCCGGCCTGAAACCGGGTGTTG | |
| | | 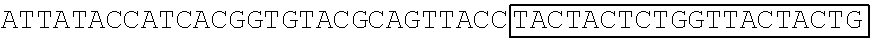 | |
| | | 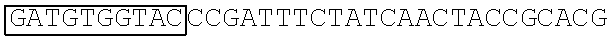 | |
| | | (Amino acid sequence) | 34 |
| | | LEVVEASP-TSIQISWDA-SSYVTHKEDV-VRYYRITYGETG-GNSPVQ | |
| | | 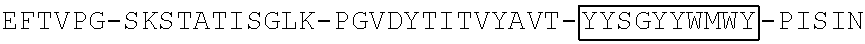 | |
| | | YRT | |

In the nucleotide sequences and amino acid sequences shown in Table 1, the sequences in the boxed parts represent FG loops.

In the present invention, the above-described DNA may be DNA consisting of a nucleotide sequence encoding the monobody polypeptide of the present invention, or DNA comprising a portion of the aforementioned nucleotide sequence and also comprising other known nucleotide sequences necessary for gene expression (e.g., a transcription promoter, an SD sequence, a Kozak sequence, a terminator, etc.), and thus, the DNA is not limited. Besides, in the nucleotide sequence encoding the monobody polypeptide of the present invention, the type of a codon is not limited, and for example, the codon generally used in fibronectin may be used, or those generally used in microorganisms such as E. coli and yeasts, or those generally used in plants, etc. may also be used. Thus, the codon can be appropriately selected or designed.

Herein, the “stringent conditions” may be any of low-stringent conditions, moderate-stringent conditions, and high-stringent conditions. The “low-stringent conditions” are, for example, conditions consisting of 5× SSC, 5× Denhardt's solution, 0.5% SDS, 50% formamide, and 32° C. The moderate-stringent conditions” are, for example, conditions consisting of 5× SSC, 5× Denhardt's solution, 0.5% SDS, 50% formamide, and 42° C. The high-stringent conditions” are, for example, conditions consisting of 5× SSC, 5× Denhardt's solution, 0.5% SDS, 50% formamide, and 50° C.

Under these conditions, it is expected that DNA with high homology can be obtained more efficiently, as the temperature is increased. However, multiple elements such as temperature, probe concentration, probe length, ionic strength, time and salt concentration may affect the stringency of hybridization, and those skilled in the art can realize similar stringency by selecting these elements, as appropriate.

Other than the above-described DNAs, DNAs capable of hybridization include DNAs having a homology (identity) of about 80% or more with the DNA encoding the monobody, when the homology (identity) is calculated by homology search software such as FASTA and BLAST, using default parameters, and further, DNAs having a homology (identity) of 85% or more, 90% or more, 95% or more, 96% or more, 97% or more, 98% or more, or 99% or more with the DNA encoding the monobody.

### (2) Recombinant Vector

In the present invention, a recombinant vector is obtained by inserting the above-described DNA of the present invetion into an appropriate vector. The method of obtaining such a recombinant vector is known (Sambrook J. et al., Molecular Cloning, A Laboratory Manual (4th edition), Cold Spring Harbor Laboratory Press (2012)). The vector, into which the DNA of the present invetion is inserted, is not particularly limited, as long as it can replicate in a host. Examples of the vector may include plasmid DNA, phage DNA, and viruses.

Examples of the plasmid DNA may include plasmids derived from E. coli, Bacillus subtilis, and yeasts. An example of the phage DNA may be lambda phage. Examples of the viruses may include adenoviruses, baculoviruses, and retroviruses.

To the recombinant vector of the present invention, a promoter, the DNA of the invention, and also, as desired, cis-elements such as an enhancer, and a splicing signal, a poly A addition signal, a ribosome binding sequence (SD sequence), a selection marker gene, a reporter gene, etc., can be linked. Besides, examples of the selection marker gene may include a dihydrofolate reductase gene, an ampicillin resistance gene, and a neomycin resistance gene. Examples of the reporter gene may include genes of a green fluorescent protein (GFP) or mutants thereof (e.g. fluorescent proteins such as EGFP, BFP, and YFP), luciferase, alkaline phosphatase, and LacZ.

### (3) Transformant and Culture

The present invention also includes a transformant that can be obtained by introducing the above-described recombinant vector of the present invention into a host so that a gene of interest can be expressed.

The host used for transformation is not particularly limited, as long as it can express a gene of interest. Exmaples of the host may include bacteria (E. coli, Bacillus subtilis, etc.), yeasts, animal cells (COS cells, CHO cells, etc.), and insect cells or insects. Mammals such as goats can also be used as hosts. The method of introducing a recombinant vector into a host is known (Sambrook J. et al., Molecular Cloning, A Laboratory Manual (4th edition), Cold Spring Harbor Laboratory Press (2012)).

When a bacterium is used as a host, the recombinant vector of the present invention is capable of autonomous replication in the bacterium, and at the same time, can comprise a promoter, a ribosome binding sequence, the DNA of the present invention, and a transcription termination sequence. Examples of the bacteria may include E. coli. An example of the promoter may be a lac promoter. As methods of introducing the vector into bacteria, various types of known introductio methods, such as, for example, a calcium ion method, may be applied.

When a yeast is used as a host, for example, Saccharomyces cerevisiae is used. In this case, the promoter is not particularly limited, as long as it can be expressed in the yeast, such as a gall promoter. Examples of the method of introducing the vector into a yeast may include an electroporation method and a spheroplast method.

Then, the above-described transformant is cultured, and a monobody polypeptide is then collected from the culture product. The term “culture product” means either (a) a culture supernatant, or (b) cultured cells, a cultured cell mass, or a crushed product thereof.

After completion of the culture, when a monobody of interest is produced inside a cell mass or inside cells, the monobody is extracted by crushing the cell mass or the cells. If the monobody of interest is produced outside the cell mass or outside the cells, the culture medium is directly used as is, or the cell mass or the cells are removed by centrifugation, etc. Thereafter, common biochemical methods used for isolation and purification of proteins, such as ammonium sulfate precipitation, gel filtration, ion exchange chromatography, affinity chromatography, hydrophobic chromatography, and reverse phase chromatography, are used alone or in combination, as appropriate, so that the monobody of interest can be isolated and purified.

The isolated and purified monobody having a dissociation constant of 5 nM or less is a synthetic anti-ubiquitin antibody and has high affinity for ubiquitin.

### 5. Detection Method and Detection Reagent

The synthetic antibody of the present invention can be used as a reagent for detecting a ubiquitin or a ubiquitinated substrate.

The method of detecting a ubiquitin or a ubiquitinated substrate comprises, for example:
(a) a step of allowing the synthetic antibody of the present invention to react with a test sample, and
(b) a step of allowing an antibody-test sample complex formed in the step (a) to react with a labeled antibody for detection.

After completion of the reaction, signals emitted from the labeled antibody are detected.

The detection method of the present invention can be generally carried out in accordance with the assay using antibodies (immunoglobulins). Examples of the detection method may include enzyme-linked immunoassay (ELISA), fluorescence immunoassay, fluorescent antibody assay, and Western blotting.

The sample used in the detection method of the present invention is not particularly limited, as long as it is a sample containing ubiquitin, such as yeasts, cultured eukaryotic cells, protein extracts derived from zebrafish or mouse tissues, which contain endogenous ubiquitin, or protein extracts derived from prokaryotic cells and cell-free system solutions, which contain recombinant ubiquitin.

The labeling substance that can be used in the present invention is not particularly limited, as long as it enables the detection of the presence of a synthetic antibody by allowing it to bind to the synthetic antibody via a physical bond, a chemical bond, etc. Specific examples of the labeling substance may include enzymes, fluorescent substances, chemiluminescent substances, biotin, avidin, and radioisotopes. More specific examples thereof may include peroxidase, alkaline phosphatase, β-D-galactosidase, biotin, avidin, and chemiluminescent substances.

The reagent for detection of a ubiquitin or a ubiquitinated substrate of the present invention is used as a kit including the synthetic antibody of the present invention. The synthetic antibody used herein may also be the above-described immobilized antibody or labeled antibody. The kit of the present invention may include various auxiliary agents in addition to these antibodies in order to utilize the kit efficiently and easily. Examples of such auxiliary agents may include buffer solutions, substrates for measuring enzyme activity when enzymes are used as labeling substances, and reaction stoppers, which are usually used in kits for immunological assay reagents. In addition, the kit of this invention can also include an instruction manual.

### 6. Method for Producing Ubiquitin or Ubiquitinated Substrate

The present invention provides a method for producing a ubiquitin or a ubiquitinated substrate.

As described above, a ubiquitin modifies a target protein by covalently binding to the target protein. Accordingly, by using the monobody (synthetic antibody) of the present invention, not only a ubiquitin and a polyubiquitin, but a ubiquitinated protein (ubiquitinated substrate), in which the target protein is modified with a ubiquitin or a polyubiquitin, can also be obtained.

For example, when a ubiquitin is conjugated to a target protein in a test sample, the above-described synthetic antibody is allowed to contact with the test sample. Since the synthetic antibody binds to the ubiquitin, the complex of the synthetic antibody and the test sample comprises the ubiquitin and a ubiquitinated protein (ubiquitinated substrate). Thus, the ubiquitin or the ubiquitinated protein is collected from the complex.

In order to collect the ubiquitin or the ubiquitinated protein from the complex, known methods such as immunoprecipitation, SDS-polyacrylamide gel electrophoresis, or Western blotting can be adopted alone or in combination as appropriate for purification or concentration, and thus, the method is not particularly limited.

### EXAMPLES

Hereinafter, the present invention will be more specifically described in the following examples. However, the scope of the present invention is not limited to these examples.

### Example 1

### Methods

### 1. Screening of Synthetic Anti-Ubiquitin Antibody Using TRAP Display Method

### (1) Preparation of Biotinylated Ubiquitin

A construct (pET21a-hUb-Cys-His6) was prepared by adding a Cys residue for biotin labeling and a 6xHis tag for purification to the C-terminus of a human-derived ubiquitin, and the construct was then purified by the E. coli expression system. Subsequently, a biotin tag was incorporated into the Cys residue using EZ-Link™ Maleimide-PEG11-Biotin (Thermo Fisher Scientific).

Specifically, 3 μL of 0.1 M TCEP was added to 30 μL of 576 μM ubiquitin, and the obtained mixture was then left at rest at room temperature for 30 minutes. (TCEP: f.c. 10 mM) TCEP was removed by gel filtration with HBST, and the concentration was determined by 20% SDS-PAGE. The ubiquitin was diluted to 300 μM, and 1.2 μL of 2.5 mM EZ-Link™ Maleimide-PEG11-Biotin was then added to 10 μL of the 300 μM ubiquitin. The obtained mixture was left at rest at room temperature for 2 hours. Thereafter, 1 μL of 50 mM β-mercaptoethanol was added to the reaction mixture, and the thus obtained mixture was left at rest at 4° C. for 1 hour. The obtained biotinylated ubiquitin was confirmed by 20% SDS-PAGE.

### (2) TRAP Display Method

FN3 proteins were translated in vitro from a small-molecule-type synthetic antibody monobody FN3 mRNA library (equivalent to 10¹³), and then, clones interacting with ubiquitin were selected using a biotinylated ubiquitin as a bait. By repeating this screening multiple times, clones binding to ubiquitin with high affinity were concentrated. Subsequently, the nucleotide sequences of the variable regions were determined using Next-Generation DNA Sequencer, and 7 different types of anti-ubiquitin monobody clones were obtained.

### 2. Measurement of Binding Affinity between Monobody and Ubiquitin

### (1) Expression and Purification of Monobody Protein in E. coli

A construct harboring a monobody, a Nus tag for solubilization, and a hexahistidine tag (His6) for purification from the N-terminus was prepared. Subsequently, a recombinant protein was expressed in E. coli, and then purified using the His6 tag. Specifically, the following procedures were carried out.

### Cloning of Plasmid DNA for Expression in E. coli

### Preparation of Inserts

PCR primers were designed from the sequences of the first and third loops of a monobody selected for each target protein, and only the clones of interest from the DNA pool obtained by koff selection were amplified by a PCR method. A PCR reaction solution (80 μL) consisting of 1× PfSH buffer, 0.2 mM dNTPs, 0.375 μM each Primer, 2.5 nM DNA pool, 2% DMSO 1/100 (v/v) and 1/60 PfSH (1406) was prepared for each target protein, and thereafter, PCT was carried out for 94° C. (1 min)−[94° C. (20 sec)−65° C. (20 sec)−72° C. (45 sec)]×15 cycles. The reaction solution obtained after the PCR reaction was treated with phenol/chloroform, and was then subjected to iProOH precipitation. The obtained precipitate was dissolved in 8 μL of 10 mM Tris-AcOH (pH 7.8), and was then analyzed by 8% Native PAGE (1:37.5) to determine the concentration.

### Cleavage of Vector

In 45 μL of a solution of 1× Cut Smart buffer, 1/100 BspQI and 1/100 CIP, 5 μL of 100 ng/μL pQCNus2-FN3B1del solution was placed and mixed, and the obtained mixture was reacted in a water bath at 37° C. for 30 minutes. Thereafter, the tube was exchanged, and the reaction was carried out again in a water bath at 37° C. for 30 minutes. After a restriction enzyme treatment, the solution was treated with phenol/chloroform, and iProOH precipitation was performed. The obtained precipitate was dissolved in 5 μL of 10 mM Tris-AcOH (pH 7.8) and was then analyzed by 1% agarose electrophoresis to determine the concentration.

### Cloning

Each insert was incorporated into the restriction enzyme-treated pQCNus2-FN3B1del by an MuPaC method⁴⁵. After the MuPac, 2 μL of the solution was added to 20 μL of chemical competent cells (High 108 HIT-JM109 J80 (Scitrove)), and the mixed solution was then left at rest on ice for 10 minutes. Thereafter, the reaction solution was heat-shocked in a water bath at 42° C. for 1 minute, and the resulting solution was again left at rest on ice for 10 minutes. Thereafter, the reaction solution was diluted with 400 μL of LB, and the resultant was then subjected to shaking culture at 37° C. for 1 hour. The culture medium that had been further 10-fold diluted with LB was spread on an LB plate containing 100 μg/mL ampicillin, and was then left at rest overnight in a thermostatic bath at 37° C. The formed colonies were pushed out, and were added to 2 mL of LB containing 100 μg/mL ampicillin, followed by performing shaking culture at 37° C. overnight. The proliferating cell mass was recovered, and plasmid DNA was then extracted using FastGene™ Plasmid Mini Kit (Nippon Genetics Co., Ltd.). The nucleotide sequence of the obtained plasmid DNA was confirmed by a Sanger method.

### Expression of Monobody Using E. coli

To 20 μL of BL21 (DE3) LysS competent cells, 0.5 μL of 50 ng/μL plasmid DNA (pQCNus2-FN3) was added, and the obtained mixture was then left at rest on ice for 10 minutes. After the reaction mixture was heat-shocked in a water bath at 42° C. for 1 minute, resulting solution was again left at rest on ice for 10 minutes. Thereafter, the reaction solution was diluted with 400 μL of LB, and the resultant was then subjected to shaking culture at 37° C. for 1 hour. Thereafter, the culture medium was spread on an LB plate containing 100 μg/mL ampicillin, 20 μg/mL chloramphenicol and 2% glucose, and was then left at rest overnight in a thermostatic bath at 37° C. Multiple colonies formed were suspended in LB, and were added to 300 mL of LB containing 100 μg/mL ampicillin, 20 μg/mL chloramphenicol and 1× ZYP. This culture medium was shaken at 25° C. for 24 hours, and was then centrifuged at 4° C. at 7000 rpm for 3 minutes to collect a cell mass. This cell mass was suspended in 30 mL of 1× IMAC B1K (-glycerol) with 120 μg/mL PMSF, and the obtained suspension was then aliquoted in 15-mL portions, which were then stored at −80° C.

### Purification of Monobody

15 mL of the suspension of cell mass was dissolved on ice water. The dissolved cell mass was crushed by subjecting it to ultrasound for 1 minute 30 seconds. To the crushed solution, 1.6 mL of glycerol was added, and they were mixed with each other by inversion, and the obtained mixture was then centrifuged at 4° C. at 13000 rpm for 10 minutes. The obtained supernatant was filtered through Minisart (registered trademark) GF Syringe Filters (sartorius) and Minisart (registered trademark) high flow Syringe Filters (sartorius). The filtrate was purified using NGC™ Chromatography Systems (BIO-LAD). At this time, Bio-Scale™ Mini Profinity™ IMAC Cartridges, 1 mL (BIO-LAD) was used as a column. After sample applying, the sample was washed with 1× IMAC B1K (+ATP) for 20 CV, and then with 1× IMAC B1 for 10 CV, and was then eluted with 1× IMAC B3. The eluted fractions having a high absorbance (280 nm) were mixed with one another, and 1 M DTT was then added to the mixture to 1 mM DTT. Thereafter, the concentration was determined by UV-vis.

### (2) Determination of Dissociation Constant by Bio-Layer Interferometry Method (BLI)

A biotinylated ubiquitin (5 nM) was immobilized on a streptavidin sensor, and the binding dissociation of an anti-ubiquitin monobody (2-fold dilution series of 0.625 to 10 nM) used as an analyte was monitored. Curve fitting was carried out using the specialized software ForteBio Data Analysis ver 10.0, and the dissociation constant (Kd) was then calculated from the calculated first-order reaction approximation curve.

### 3. Western Blot Analysis of Ubiquitinated Substrate Using Cell Extract

### (1) Purification of GST Monobody and Biotin Labeling

A construct (pGST-TEV-GGGS-monobody) was prepared by fusing a glutathione-S-transferase (GST) tag, a TEV protease recognition sequence, and a GGGS linker to the N-terminus of a monobody, and it was then transformed into the E. coli strain BL21 (DE3).

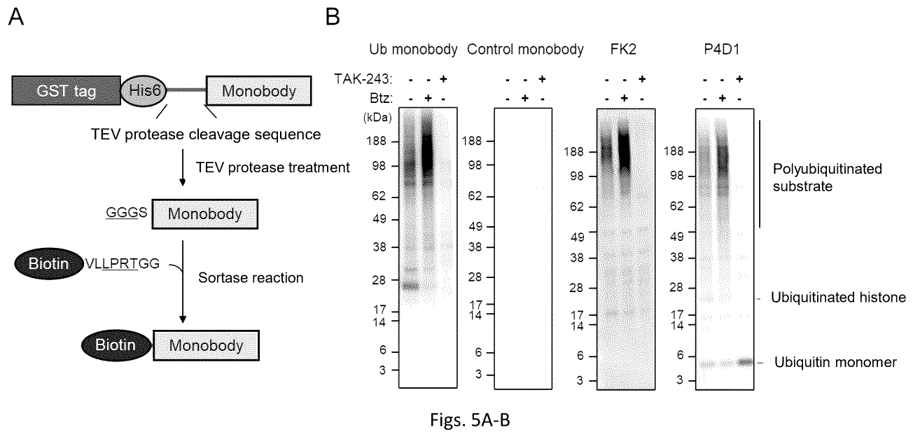

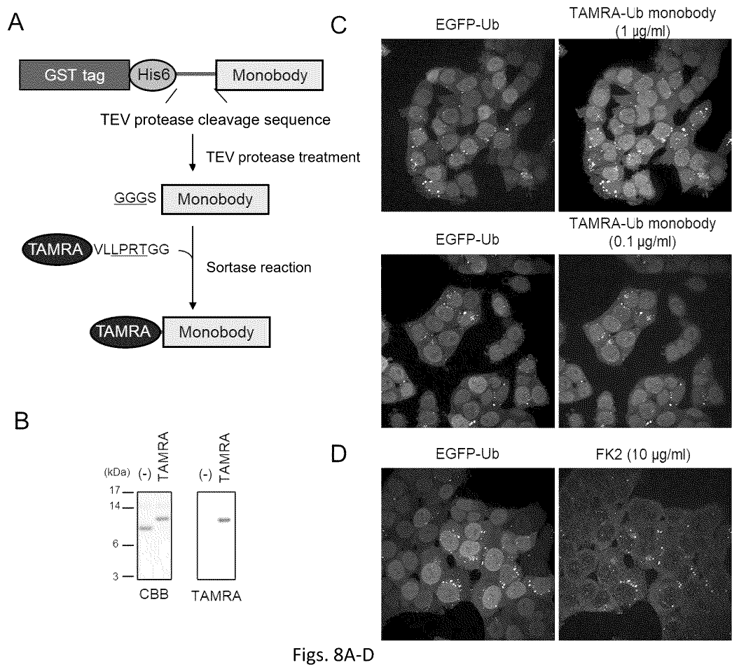

The DNA sequences and amino acid sequences of the monobody for biotin and TAMRA labeling (used for Western blot analysis (FIG. 5) and immunostaining (FIG. 8)) are shown in Table 2.

TABLE 2

| | Sequence | SEQ ID No. |
|---|---|---|
| DNA | CTGGAAGTTGTGGAAGCCAGCCCGACGAGCATTCAGATTTCTT | 35 |
| Sequence | GGGACGCTGCTTCTTTCGGTTTCACTCATATGGACAAAGTTCG | |
| | CTACTATCGCATTACCTATGGCGAAACCGGCGGTAACAGTCCG | |
| | GTCCAGGAATTTACGGTGCCGGGTTCAAAATCGACCGCGACGA | |
| | TTTCCGGCCTGAAACCGGGTGTTGATTATACCATCACGGTGTA | |
| | 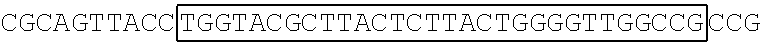 | |
| | ATTTCTATCAACTACCGCACGGAAATTGATAAA | |
| Amino acid | LEVVEASP-TSIQISWDA-ASFGFTHMDK-VRYYRITYGETG- | 36 |
| sequence | 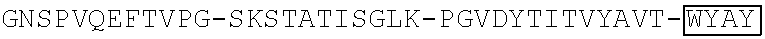 | |
| | 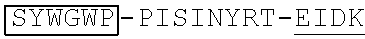 | |

In the above sequences, the framed sequences represent FC loops, and the underlined sequences represent C-terminus stabilized sequences.

Subsequently, the construct was cultured in an LB medium containing 100 uM ampicillin at 37° C., and the expression of a GST tag-fused monobody was induced with 0.2 mM IPTG, followed by purification using the GST tag. The GST tag-fused monobody was digested with TEV protease (Promega, ProtTEVPlus). The N-terminal sequence GGGS of the monobody generated by protease digestion was utilized, and a peptide for biotin labeling (Biotin-PEG12-VLLPRTGG; synthesized by Eurofin) was ligated according to a Sortase reaction (20 uM monobody, 1 mM biotin-labeling peptide, 8.3 uM Sortase 5Y, 50 mM Tris-HCl, pH 7.5, 150 mM NaCl, 5 mM CaCl2; 37° C., 1 hour). Subsequently, using a desalting column (Sephadex G50, Cytiva), unreacted peptides were removed, and at the same time, the buffer was replaced with PBS. Besides, as a control, a wild-type monobody was used.

### (2) Preparation of Cell Extract

Human colon cancer-derived HCT116 cells (ATCC, CCL-247) were cultured in Dulbecco's Modified Eagle Medium (DMEM) (SIGMA) supplemented with 10% fetal bovine serum (Biowest), 1 mM sodium pyruvate (Thermo Fisher Scientific) and non-essential amino acids (Thermo Fisher Scientific) at 37° C. in an incubator containing 5% CO₂. The cells were cultured in the DMEM medium containing 1 uM ubiquitin E1 inhibitor TAK-243 (Active Biochem) and 1 uM proteasome inhibitor bortezomib (LC Laboratories) for 3hours. Subsequently, the cells were washed with PBS, lysed in Lysis buffer (50 mM Tris-HCl, pH 7.5, 150 mM NaCl, 1% SDS), and then centrifuged (15,000 rpm, 20 min) to obtain a soluble fraction. The protein concentration was determined using BCA assay kit (Thermo Fisher Scientific). LDS sample buffer (containing 2% β-mercaptoethanol as a reducing agent) was added in an amount of 5 ug to one sample, and the sample was incubated at 70° C. for 10 minutes and subjected to Western blot analysis.

### (3) Western Blot Analysis

The above sample was electrophoresed on 4%-12% NuPAGE Bis-Tris gel (Thermo Fisher Scientific) in MES buffer (Thermo Fisher Scientific). See Blue2 (Thermo Fisher Scientific) was used as a molecular weight marker. After the electrophoresis, a protein was transferred onto a PVDF membrane (Millipore) using XCell II Blot Module (Thermo Fisher Scientific). The PVDF membrane after the transcription was then stained with Ponceau S to confirm that the amount of protein in each lane was same. Subsequently, the blot for biotinylated monobody was blocked with PBST containing 3% BSA, and the blot for IgG-type anti-ubiquitin antibody was blocked with TBST containing 5% nonfat milk for 30 minutes. As primary antibodies, biotin-labeled anti-ubiquitin monobody ((2) above), ubiquitin antibody P4D1 (sc-8017, SantaCruz), and polyubiquitin monoclonal antibody FK2 (0918-2, Nippon Biotest Inc.) were used, and incubation was carried out at room temperature for 2 hours. As a secondary antibody, Streptavidin-HRP conjugate (Invitrogen, SA10001) or Mouse IgG HRP conjugate (Promega, W402B) was used. After the cells were washed with TBST several times, the cells were chemiluminescent with ECL Prime detection reagent (Cytiva), and were then photographed with a FUSION FX7 imaging system (Vilber-Lourmat).

### 4. Immunoprecipitation of Ubiquitinated Substrate Using Cell Extract

### (1) Purification of Halo Tag-Fused Monobody

A construct (pHalo-TEV-GGGS-monobody) was prepared by fusing a hexahistidine tag (His6), a Halo tag, a TEV protease recognition sequence, a GGGS linker, and a monobody with one another from the N-terminus, and the prepared construct was then transformed into the E. coli strain BL21 (DE3). Subsequently, the cells were cultured in an LB medium containing 100 uM ampicillin at 37° C., and the expression of a Halo tag-fused monobody was induced with 0.2 mM IPTG, and was purified using a His6 tag. Clone C was selected as an anti-ubiquitin monobody, and a wild-type monobody was used as a control.

### (2) Preparation of Cell Extract

Semi-confluent HCT116 cells were washed with PBS, and thereafter, the cells were dissolved in TNE buffer (50 mM Tris-HCl, pH 7.5, 150 mM NaCl, 1% NP-40, and 0.5 mM EDTA) that contained various protease inhibitors (10 mM MG132, 10 mM PR619, 10 mM iodoacetamide, 1× complete protease inhibitor cocktail (Roche, EDTA-free)). Thereafter, centrifugation (15,000 rpm, 20 min) was performed to obtain a soluble fraction. Subsequently, the protein concentration was determined using BCA assay kit (Thermo Fisher Scientific).

### (3) Immunoprecipitation

A Halo tag-fused monobody was immobilized on Magne HaloTag Beads (Promega) at a concentration of 0.2 nmol for one sample. As IgGs, control IgG (normal mouse IgG, 140-09511, FUJIFILM Wako Pure Chemical Corporation), anti-polyubiquitin monoclonal antibody FK2 (0918-2, Japan Biotest,), anti-ubiquitin antibody P4D1 (sc-8017, Santa Cruz) were immobilized at a concentration of 0.2 nmol each on Dynabeads Protein G (Thermo Fisher Scientific). Subsequently, 200 ul (100 ug) of HCT116 cell extract was added to each sample and they were then mixed by inversion at 4° C. overnight. After the mixture was washed three times with TNE buffer, 1× LDS sample buffer for electrophoresis (containing 2% β-mercaptoethanol as a reducing agent) was added to the beads, and the thus obtained mixture was then heated at 70° C. for 10 minutes, so that an immunoprecipitated was eluted.

### (4) Western Blot Analysis

Western blot analysis was performed in the same manner as that described in the above section 3(3). The antibody used was HRP-labeled anti-ubiquitin antibody P4D1-HRP (sc-8017 HRP, Santa Cruz).

### 5. Absolute Quantification of Ubiquitin Chain Using Mass Spectrometry

### (1) In-Gel Digestion

The above-described immunoprecipitate was electrophoresed on 4%-12% NuPAGE Bis-Tris gels (Thermo Fisher Scientific) in MES buffer (Thermo Fisher Scientific) for about 2 cm, and was then stained with CBB (Bio-Safe Commassie, Bio-Rad). After the gel was washed with Milli-Q water, a gel of 62 kDa or more was cut out using a molecular weight marker as an indicator, and the gel was cut into 1 mm squares. The gel pieces were collected in an Eppendorf tube and were washed with 50 mM ammonium bicarbonate and 30% acetonitrile (ACN) for 2 hours, and then with 50 mM ammonium bicarbonate and 50% ACN for 1 hour, and were dehydrated in 100% ACN for 15 minutes. Subsequently, a trypsin solution (20 ng/ml Trypsin Gold, Promega, 50 mM ammonium bicarbonate, and 5% ACN) was added to the resultant, which was then digested at 37° C. overnight. The digested peptide was extracted twice with 0.1% trifluoroacetic acid and 70% acetonitrile.

### (2) Absolute Quantification of Ubiquitin Chain

Into the above extracted peptide, a stable isotope-labeled ubiquitin chain-derived peptide mixture (25 fmol/measurement) serving as an internal standard (F. Ohtake, H. Tsuchiya, K. Tanaka, Y. Saeki, Methods to measure ubiquitin chain Methods Enzymol 618, 105-133 (2019) was mixed, and the obtained mixture was then concentrated by vacuum centrifugation. Thereafter, 20 ul of 0.1% TFA containing 0.05% hydrogen peroxide was added to the concentrate, and the thus obtained mixture was then incubated at 4° C. overnight. It is to be noted that the hydrogen peroxide was added to completely oxidize methionine residues in the peptide.

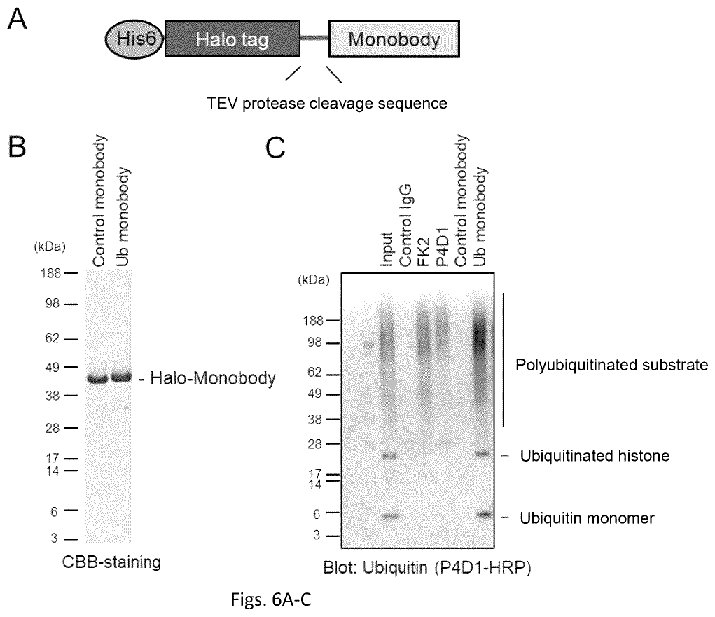

Subsequently, the sample was analyzed using liquid chromatography-tandem mass spectrometry device (Easy nLC 1200 with Orbitrap Fusion LUMOS, Thermo Fisher Scientific). A C18 analytical column (Aurora Series Emitter Column, AUR2-25075C18A, IonOpticks) was used for separation with a gradient of 50 minutes (solvent A, 0.1% formic acid; solvent B, 80% ACN/0.1% formic acid). The MS/MS spectrum of the peptide derived from the ubiquitin chain was obtained as a target, and the abundance of the peptide was calculated based on the area under the integration curve (AUC) of selected fragment ions, using PinPoint software 1.3 (Thermo Fisher Scientific). The targeted peptide sequence is shown in FIG. 6A.

### 6. Immunofluorescent Staining

### (1) Fluorescent Labeling of Monobody

The GST tag-fused monobody prepared in the above section 3 was digested with TEV protease (ProtTEVPlus, Promega). Utilizing the N-terminal sequence GGGS of the monobody generated by the protease digestion, a TAMRA-labeled peptide (TAMRA-PEG12-VLLPRTGG, synthesized by Eurofin) was ligated by a Sortase reaction (10 uM monobody, 500 uM TAMRA-labeled peptide, 8.3 uM Sortase 5Y, 50 mM Tris-HCl, pH 7.5, 150 mM NaCl, and 5 mM CaCl2; 37° C., 1 hour). Subsequently, unreacted peptides were removed using a desalting column (Sephadex G50, Cytiva), and at the same time, the buffer was replaced with TBST.

### (2) Immunostaining

HCT116 cells stably expressing EGFP-fused ubiquitin (S. Yasuda et al., Stress- and ubiquitylation-dependent phase separation of the proteasome. Nature 578, 296-300 (2020).) were cultured in a 35-mm glass bottom dish (MatTek), and thereafter, formation of ubiquitin-positive aggregates was induced by a puromycin treatment (5 ug/ml, 2 hours). After immobilization in PBS containing 4% paraformaldehyde (Thermo Fisher Scientific) for 15 minutes, the cells were treated with MeOH (Wako) at −20° C. for 5 minutes. Subsequently, after blocking in PBS containing 1% FBS, the cells were stained with a TAMRA-labeled anti-ubiquitin monobody (0.1 ug/ml or 1 ug/ml) or an anti-polyubiquitin monoclonal antibody FK2 (0918-2, 10 ug/ml, NIPPON BIO-TEST LABORATORIES INC.) for 1 hour. In the case of the FK2 antibody, Alexa568-labeled anti-mouse IgG antibody (A-11031, Thermo Fisher Scientific) was used as a secondary antibody, and the cells were stained for 1 hour. Subsequently, the cells were stained with PBS containing DAPI (Thermo Fisher Scientific) for 15 minutes, and an anti-fading reagent (SlowFade Gold, Thermo Fisher Scientific) was added to the resulting cells, and the cells were then sealed with a cover strip glass.

### (3) Fluorescence Microscope Analysis

Images were acquired with an inverted fluorescence microscope IX73 (Olympus Corporation) equipped with a CSU-X1 spinning disk fluorescence scanner (Yokogawa Electric Corporation), PlanApo100× OTIRF 1.45NA (Olympus Corporation), ORCA-Flash4.0 V3 Digital CMOS camera (Hamamatsu Photonics K.K.) Images were acquired with an inverted fluorescence microscope IX73 (Olympus Corporation). Using MetaMorph software (Molecular Devices), 16 fluorescence images (DAPI, EGFP, and TAMRA/Alexa568 channels) were acquired at 0.2 um each in the Z direction, and were then superimposed on one another to obtain Maximum projection images.

### Results and Discussion

### 1. Acquisition of Anti-Ubiquitin Monobody

Aiming to acquire a synthetic antibody monobody that binds to a ubiquitin monomer with high affinity, a synthetic antibody monobody (FN3) random library was used to perform screening according to a TRAP presentation method (T. Kondo et al., Antibody-like proteins that capture and neutralize SARS-CoV-2. Sci Adv 6 (2020).). The present screening was repeated for a total of 5 cycles, and the concentrated monobody mRNA clones were analyzed using a Next-Generation DNA Sequencer, so as to obtain a total of 7 anti-ubiquitin monobody clones (FIG. 3).

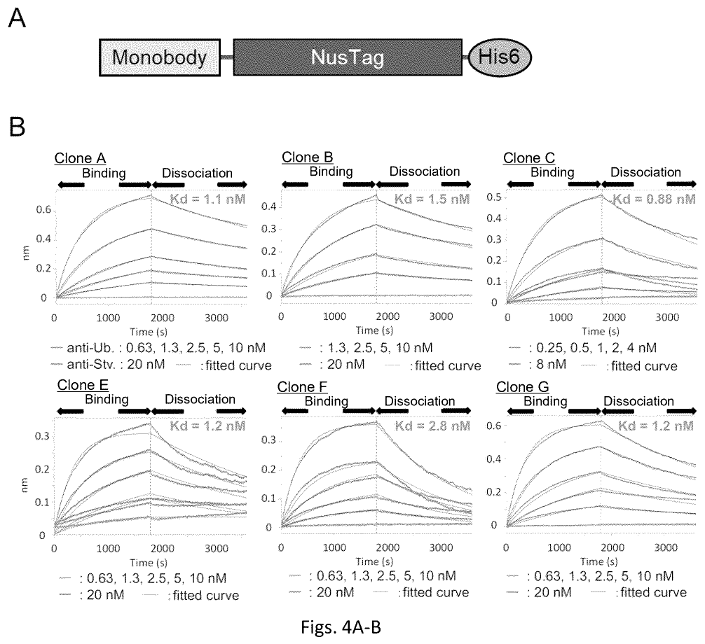

Subsequently, recombinant proteins of the anti-ubiquitin monobody were prepared by an E. coli expression system, and their interaction with ubiquitin was analyzed by the bio-layer interferometry method (BLI). As a result, the dissociation constant (Kd) was determined to be 0.88 nM to 2.8 nM for 6 clones except clone D, and it became clear that all clones bind to ubiquitin very strongly (FIG. 4).

An FN3 monobody library used for screening involves randomization of the amino acid sequences of two loops, and this library is composed of 10¹³ different clones (i.e., 10 trillion different clones). In the fifth round of NGD analysis, the emergence percentage of the same clones is 0.7% to 2.2%, which means that clones having the same sequence are identified multiple times. Therefore, it is highly likely that monobodies having affinity for ubiquitin could be obtained from the library without leakage (FIG. 3B, right).

Although P4D1 and FK2 are commonly used as existing IgG-type anti-ubiquitin mouse monoclonal antibodies in Western blot analysis or immunoprecipitation experiments, which sequence of ubiquitin is recognized as an epitope or the binding affinity with ubiquitin has not been evaluated. It is worth mentioning that the obtained anti-ubiquitin monobody has a Kd of around 1 nM, and has an affinity that is the same level as or higher than that of a general IgG-type antibody.

### 2. Western Blot Analysis Using Anti-Ubiquitin Monobody

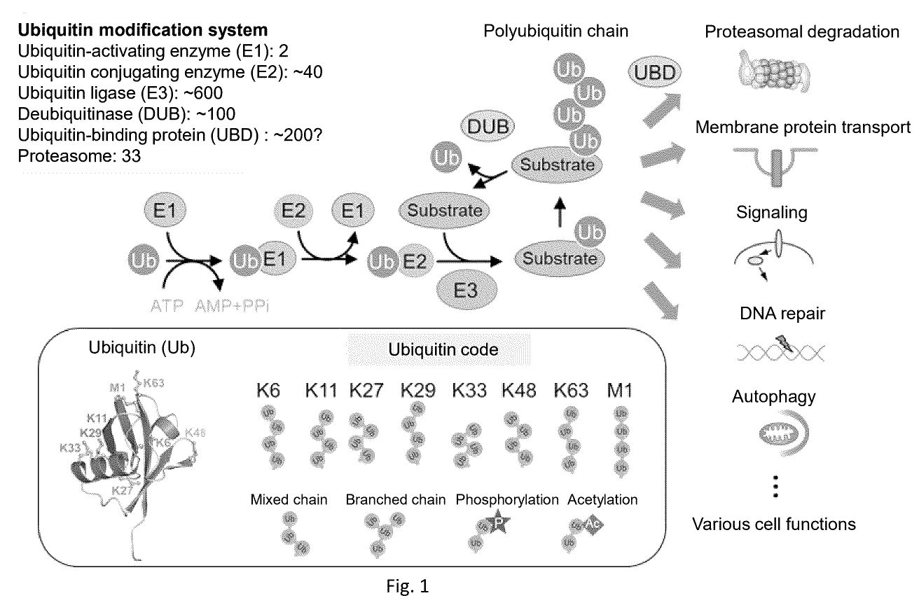

In a cell, ubiquitin is present in any state of a monomer, a ubiquitin that is thioester-bound to ubiquitinating enzymes (E1 and E2), a monoubiquitinated substrate, and a polyubiquitinated substrate (FIG. 1). Although to what extent the monoubiquitinated substrates exist in the cell is unknown, it is known that histone H2A is highly monoubiquitinylated. Thus, in the Western blot analysis of a cell extract treated with a reducing agent, a polyubiquitinated substrate smear, a ubiquitin monomer, and monoubiquitinylated histone H2A are usually detected.

Also, it is known that inhibition of the proteasome causes polyubiquitinated proteins to accumulate without degradation, and that ubiquitinylated histone is deubiquitinated and reduced. On the other hand, it is known that, when the ubiquitin-activating enzyme E1 is inhibited, the deubiquitination reaction becomes dominant, polyubiquitinated substrates are decreased, and ubiquitin monomers are increased.

Thus, using extracts of human colon cancer-derived HCT-116 cells that are treated with a proteasome inhibitor or an E1 inhibitor under normal culture conditions, the applicability of an anti-ubiquitin monobody to Western blot analysis was studied by comparing with the existing IgG-type antibodies P4D1 and FK2 (FIG. 5).

As a result, in the case of P4D1, a polyubiquitinated substrate smear and ubiquitin monomers were detected, and a fluctuation by the proteasome inhibitor and the E1 inhibitor was also observed. In the case of FK2, only the polyubiquitinated substrate was detected. On the other hand, no ubiquitin monomers were detected in the anti-ubiquitin monobody, but bands derived from a polyubiquitinated substrate smear and monoubiquitinylated histone were detected with high sensitivity (FIG. 5B, Ub monobody).

The reason why ubiquitin monomers are not detected in the anti-ubiquitin monobody is that the hydrophobic patches of ubiquitin recognized by the anti-ubiquitin monobody may be used for interaction with the PVDF membrane. In conclusion, although the existing IgG-type anti-ubiquitin antibody P4D1 is dominant when a ubiquitin monomer and a polyubiquitinated substrate are to be detected by Western blot analysis, the anti-ubiquitin monobody is dominant when the dynamics of a monoubiquitinylated histone and a polyubiquitinated substrate are to be detected.

### 3. Immunoprecipitation with Anti-Ubiquitin Monobody

Immunoprecipitation of a ubiquitin in a cell extract was performed using a Halo tag-fused anti-ubiquitin monobody and an IgG-type anti-ubiquitin monoclonal antibody (FIG. 6C). As a result of the Western blot analysis of the immunoprecipitate, it was found that both FK2 and P4D1 were able to immunoprecipitate polyubiquitinated substrates, but that the amount of the immunoprecipitate by the anti-ubiquitin monobody was the highest. Furthermore, only the anti-ubiquitin monobody was able to immunoprecipitate ubiquitin monomer and monoubiquitinylated histone.

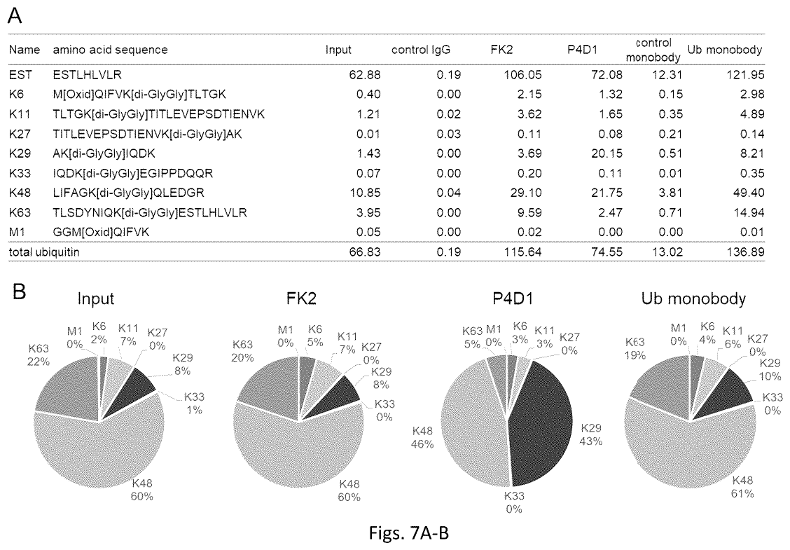

Subsequently, the composition of each polyubiquitin chain contained in the immunoprecipitate was absolutely quantified using a mass spectrometer (FIG. 7). In the present method, characteristic peptides generated by digesting 8 types of polyubiquitin chains with trypsin are analyzed using a high-resolution mass spectrometer, and the polypeptides are absolutely quantified by spiking stable isotope-labeled peptides as internal standards (F. Ohtake, H. Tsuchiya, K. Tanaka, Y. Saeki, Methods to measure ubiquitin chain length and linkage. Methods Enzymol 618, 105-133 (2019).).

As a result, the total amount of ubiquitin contained in the immunoprecipitate of the anti-ubiquitin monobody was found to be larger than that of FK2 or P4D1 (FIG. 7A).

Subsequently, the composition of the 8 types of ubiquitin chains contained in the immunoprecipitate was analyzed, and as a result, it was found that the percentage of the ubiquitin chains in the cell extract and those in the immunoprecipitates by the FK2 and the anti-ubiquitin monobody were almost identical to each other (FIG. 7B). In other words, it was found that the FK2 and the anti-ubiquitin monobody can bind to major ubiquitin chains without bias, and that they are excellent as probes for immunoprecipitation.

In addition to IgG-type anti-ubiquitin antibodies, TUBE, an artificial protein, has also been commonly used for pulling down polyubiquitinated substrates from cells, but in principle, TUBE could bind only weakly to monoubiquitinated substrates. However, TUBE can only weakly interact with monoubiquitinated substrates (R. Hjerpe et al., Efficient protection and isolation of ubiquitylated proteins using tandem ubiquitin-binding entities. EMBO reports 10, 1250-1258 (2009).). Thus, the anti-ubiquitin monobody has dominance in order to pull down all ubiquitinated substrates including monoubiquitinated substrates from the cell extract, and it is expected to be used for proteome analysis, etc. of monoubiquitinated substrates in the future.

### 4. Immunofluorescent Staining with Anti-Ubiquitin Monobody

The main intended use of anti-ubiquitin antibodies may be spatial detection of ubiquitin by immunostaining, besides immunoprecipitation. However, since the reactivity to a ubiquitin and a ubiquitinated substrate varies depending on the immobilization method and the antibody used, intracellular precise localization of ubiquitin is not clear.

Hence, the immunostaining of the IgG-type anti-ubiquitin antibody FK2 and the anti-ubiquitin monobody was performed using cultured cells stably expressing EGFP-fused ubiquitin (FIG. 8). Since the EGFP-fused ubiquitin is uniformly observed in the cytoplasm and the nucleoplasm, the immunostaining was carried out under conditions in which formation of ubiquitin-positive aggregates was induced by a puromycin treatment. As a result, the anti-ubiquitin monobody had a staining image almost identical to the fluorescent signals of the EGFP ubiquitin (FIG. 8C). On the other hand, in the case of the FK2, ubiquitin-positive aggregates were detected in the cytoplasm, but fluorescent signals were not detected in the nucleus (FIG. 8D).

It is unknown whether the EGFP-fused ubiquitin accurately reflects intracellular localization of ubiquitinated substrates, since the size of the EGFP tag is large. However, at least in the present analysis, it was suggested that the anti-ubiquitin monobody was able to detect intracellular localization of a ubiquitin and a ubiquitinated substrate more accurately than the IgG-type anti-ubiquitin antibody FK2. This may be because of the smaller size of the anti-ubiquitin monobody (approximately 10 kDa, and 2 to 3 nm in diameter), which facilitates its penetration into the cell structure.

### Conclusion

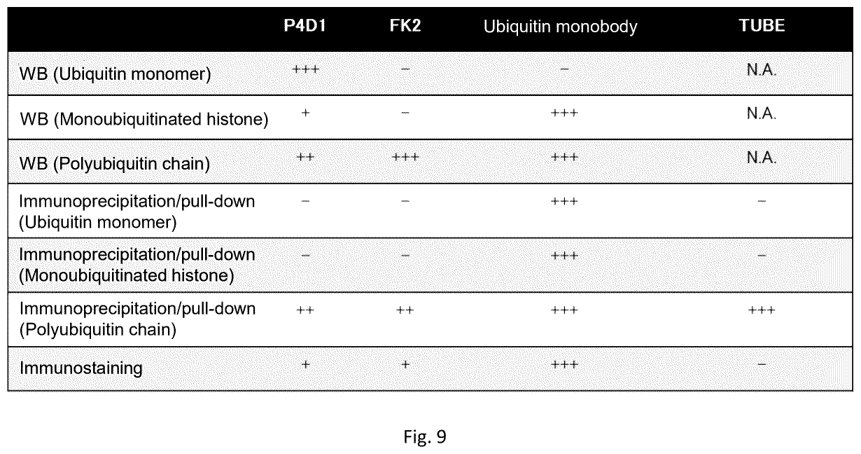

Finally, the intended use of each ubiquitin antibody and probe are summarized in a table (FIG. 9).

The anti-monoubiquitin monobody is an excellent probe that can be used in all of Western blot analysis, immunoprecipitation, and immunostaining. In particular, in immunoprecipitation experiments, the anti-monoubiquitin monobody is the only probe that can efficiently capture endogenous ubiquitin monomers and monoubiquitinated substrates in cells, and it is also an excellent probe for fluorescent immunostaining. Moreover, since the gene sequence of the anti-ubiquitin monobody has been determined, the protein can be obtained in a large amount by the E. coli expression system, and chemical modifications such as fluorescent labeling can be easily performed in vitro. Because of its small size, the monobody is expected to be sufficient for the tissue staining of human tissue sections. Furthermore, the function inhibition, etc. of ubiquitin modification by intracellular expression of the monobody is also interesting. Therefore, it can be said that the anti-ubiquitin monobody developed by the present invention is an excellent chemical tool that can be widely applied to future ubiquitin research.

### Example 2

### Cloning of Plasmid DNA for E. coli Expression

### Preparation of Insert

PCR primers were designed from the nucleotide sequence of pQCSoHis-FN3, and the FN3 gene was amplified by a PCR method. A PCR reaction solution consisting of 1× KOD one muster mix, 0.5 μM each primer, and 2.5 μg/μL plasmid DNA was prepared, PCT was carried out for [95° C. (10 sec)−55° C. (5 sec)−68° C. (5 sec)]×35 cycles. The solution obtained after the PCR reaction was separated by electrophoresis using 1% agarose gel, and was then extracted from the agarose gel using FastGene Gel/PCR Extraction Kit (Nippon Genetics Co., Ltd.).

### Cleavage of Vector

Plasmid DNA pGEX6p1 (2 μg) was mixed with 1× K buffer (TAKARA), BamHI, and XhoI. The mixed solution was left at rest in a thermostatic bath at 37° C. for 3 hours, and the plasmid DNA was cleaved. The cleaved plasmid DNA was separated by electrophoresis using 1% agarose gel, and was then extracted from the agarose gel using FastGene Gel/PCR Extraction Kit (Nippon Genetics Co., Ltd.).

### Subcloning

The insert amplified by PCR, the plasmid DNA pGEX6p1 treated with the restriction enzymes BamHI and XhoI, and 1× Gibson assembly muster mix (NEB) were mixed with one another to prepare 2 μL of a reaction solution. The reaction solution was reacted at 50° C. for 1 hour, and thereafter, 20 μL of chemical competent cells (DH5α) were added to the reaction mixture, and the obtained mixture was then left at rest on ice for 10 minutes. The reaction mixture was heat-shocked in a water bath at 42° C. for 45 second, and was then left at rest again on ice for 10 minutes. Thereafter, E. coli transformed by the heat shock method were spread on an LB plate containing 100 μg/mL ampicillin, and were then left at rest in a thermostatic bath at 37° C. overnight. The formed colonies were pushed out, and were added to 5 mL of an LB medium containing 100 μg/mL ampicillin, followed by performing shaking culture at 37° C. overnight. The proliferating cell mass was recovered, and plasmid DNA was then extracted using FastGeneTM Plasmid Mini Kit (Nippon Genetics Co., Ltd.). The nucleotide sequence of the obtained plasmid DNA was confirmed by a Sanger method.

### Expression of Monobody Using E. coli

To 20 μL of BL21 Rosetta (DE3) competent cells, 0.5 μL of 100 ng/μL plasmid DNA (pGEX6p1 GST-FN3) was added, and the plasmid DNA was introduced into E. coli according an electroporation method. The E. coli transfected with the plasmid DNA were spread on an LB plate containing 100 μg/mL ampicillin, and were then left at rest in a thermostatic bath at 37° C. overnight. Multiple colonies formed were suspended in LB, and the suspension was then added to 2.5 L of LB containing 100 μg/mL ampicillin, followed by performing shaking culture at 37° C. with shaking, until the optical density of 600 nm became 0.8. To this culture medium, isopropyl thiogalactoside with a final concentration of 0.1 mM was added, and the obtained mixture was then shaken at 15° C. overnight. Thereafter, the reaction mixture was centrifuged at 4° C. at 7000 g for 10 minutes to recover a cell mass.

### Purification of Monobody-Ubiquitin Complex

To the cell mass collected as a precipitate, 80 mL of Lysis buffer (50 mM Tris-HCl pH 8.0, 150 mM NaCl, 1 mM DTT, 0.5% Triton-X100) was added and was suspended on ice water. The dissolved cell mass was sonicated on ice water for 3 minutes, and the sonication was repeated three times to crush the cell mass. The crushed solution was centrifuged at 4° C. at 17,000 rpm for 60 minutes. The obtained supernatant was purified by Glutathione Sepharose 4B Fast Flow (GE Healthcare). The concentration of the purified protein was measured using the microabsorption spectrophotometer Nanodrop 2000 (Thermo). Ubiquitin and HRV3C were added to the purified protein, and the mixture was then dialyzed at 4° C. overnight. The dialyzed sample was passed through the anion exchange column Resource Q, and the pass-through fraction was then concentrated by the centrifugal ultrafiltration filter Amicon Ultra-15 10K (Merck). The concentrated sample was passed through the gel filtration column Superdex 75 Hiload, and the complex fraction was concentrated up to 10 g/L with Amicon Ultra-15 10K (Merck). The concentrated complex protein solution was aliquoted into a crystallization solution of the Crystallization Screening 10 kit (960 conditions), and was then left at rest at 20° C. Crystals were obtained from the crystallization solution with a composition of 0.2 M Potassium sodium tartrate tetrahydrate and 20% w/v Polyethylene glycol 3,350 for 1 week. The obtained crystals were immersed in a cryoprotectant solution containing 30% ethylene glycol, and were frozen in liquid nitrogen. The frozen crystals were subjected to a diffraction measurement with SPring-8BL45XU. The diffraction data were processed by XDS, the molecular replacement of the ubiquitin and the monobody was carried out by Molrep of CCP4, followed by refinement with refmac5 and Phenix.refine, and the final structure was determined.

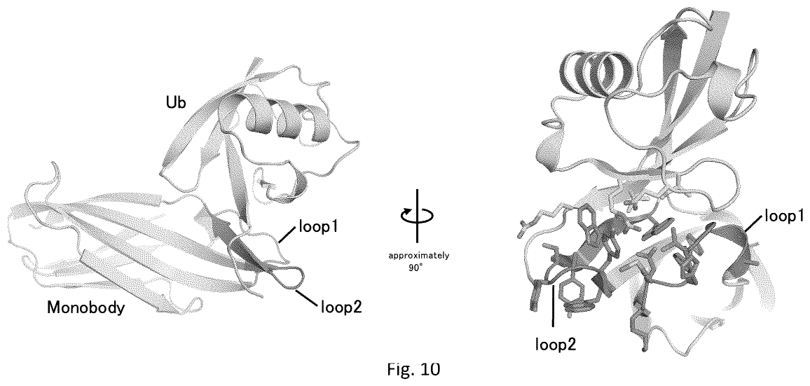

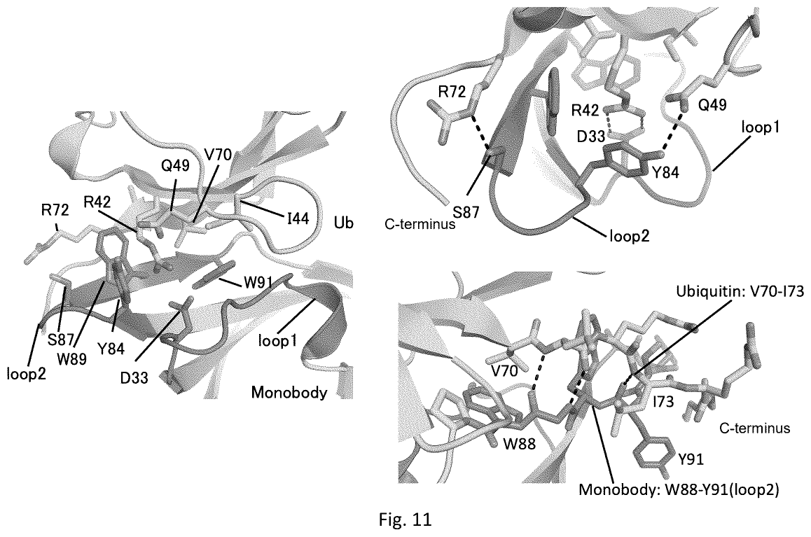

The results are shown in FIG. 10 and FIG. 11.

FIG. 10 and FIG. 11 show that the two loops of the Monobody recognize the hydrophobic patch and C-terminal region around 144 of the ubiquitin. In addition, it was shown that the Monobody recognizes the ubiquitin loops and residues spanning multiple β-strands, and that the Monobody recognizes the conformation of the ubiquitin.

### Example 3

### Selective Inhibition of Polyubiquitination

### (1) Purpose

As shown in FIG. 1, it has been elucidated that the ubiquitination reaction is caused by the enzymatic cascade of ubiquitin-activating enzyme E1, ubiquitin transferase E2,and ubiquitin ligase E3. In this reaction, peptide bonds are formed with the C-terminal carboxyl groups of the ubiquitin, and the ε-amino groups or the main chain amino groups of the lysine residues in the substrate protein. The ubiquitination reaction is repeatedly carried out on the ubiquitin, so as to form a polyubiquitin chain to which many ubiquitins bind. This reaction is referred to as polyubiquitination.

Since the amino acid sequence of the ubiquitin comprises N-terminal methionine residues and lysine residues at 7 sites, when polyubiquitination is carried out, polymers are formed in various ways (FIG. 1). To date, as modes, homogeneous chains of various lengths, branched chains, even mixed chains, etc. have been discovered. It has been known that the ubiquitin uses properly its complex chains, so that it is involved not only in proteasomal degradation, but also in various important processes in vivo, such as DNA repair, signaling, and autophagy. However, the entire biological functions of these ubiquitin chains have not yet been fully elucidated. In addition, it has been pointed out that the ubiquitin system is closely related to many diseases so far. Therefore, it is considered that clarification of the functions of various ubiquitin chains and regulation of their functions will be very important issues for the development of medicine in the future.

To date, IgG antibodies, peptides, small molecules, etc., which are specific to ubiquitin chains, have been developed as methods of inhibiting the function of ubiquitin chains and polyubiquitination reactions, but these methods have been problematic in terms of binding affinity, binding specificity, ease of production, and the like.

Thus, in the present example, the development of a technique of allowing a synthetic antibody to bind to a monoubiquitin, so as to inhibit the formation of specific polyubiquitin chains, has been attempted.

### (2) Polyubiquitination Inhibition Assay

### Confirmation of Inhibition of K48 Chain

50 mM Tris-HCl, 7.5 mM NaCl, 2 mM MgCl₂, 2 mM ATP, 20 μM ubiquitin, 2 μM E2 (E2-25K), 0.1 μM E1, and 24 μM Monobody were prepared, and the obtained mixture was left at rest in a water bath at 37° C. for 1 hour. After the reaction, 5 μL of 2× SDS Loading buffer was added to 5 μL of the reaction solution, and 9 μL of the mixed solution was placed on 12% Bis-Tris gel, and thereafter, SDS-PAGE was carried out using MES buffer as a phoresis buffer.

### Confirmation of Inhibition of K63 Chain

50 mM Tris-HCl, 7.5 mM NaCl, 2 mM MgCl₂, 2 mM ATP, 20 μM ubiquitin, 2 μM E2 (Ubc13), 2 μM E2 (MMS2), 0.1 μM E1, and 24 μM Monobody were prepared, and the obtained mixture was left at rest in a water bath at 37° C. for 1 hour. After the reaction, 5 μL of 2× SDS Loading buffer was added to 5 μL of the reaction solution, and 9 μL of the mixed solution was placed on 12% Bis-Tris gel, and thereafter, SDS-PAGE was carried out using MES buffer as a phoresis buffer.

### (3) Results

In the present example, using the K48 chain and the K63 chain, for which an in vitro synthetic system has been established, whether each Monobody inhibits the polyubiquitination reaction was examined.

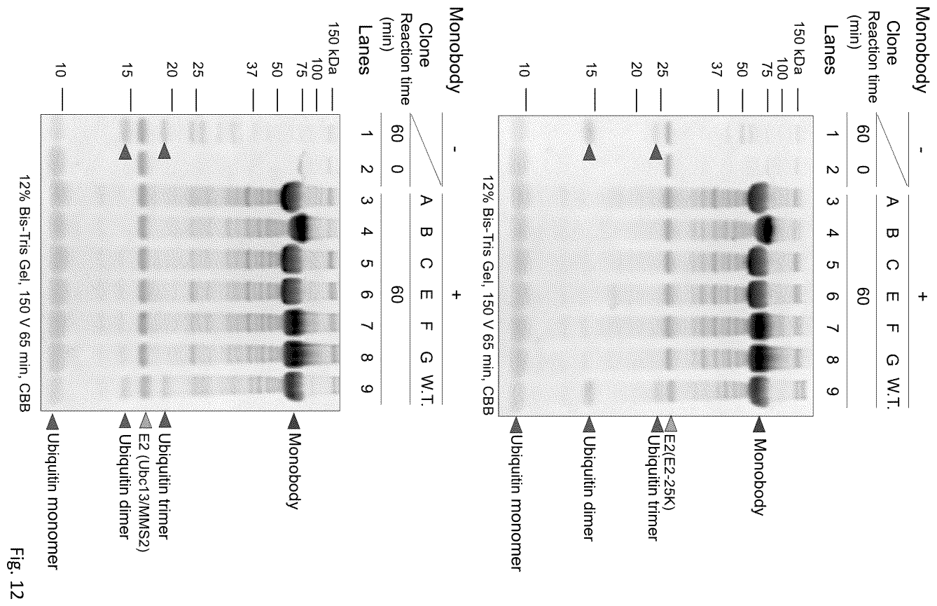

As a result, all synthetic antibodies whose binding ability had been confirmed inhibited the formation of both the K48 chain and the K63 chain (FIG. 12). In addition, when a wild-type Monobody (FN3), which does not recognize ubiquitin, was added, a band of polyubiquitin was observed. From these results, it was found that the binding of each Monobody to a ubiquitin enables inhibition of polyubiquitination.

### Sequence Listing Free Text

SEQ ID Nos: 4 to 22: Synthetic peptides
SEQ ID Nos: 23, 25, 27, 29, 31, 33, and 35: Synthetic DNAs
SEQ ID Nos: 24, 26, 28, 30, 32, 34, and 36: Synthetic polypeptides

1. A synthetic anti-ubiquitin antibody, comprising a fibronectin type III polypeptide, wherein the synthetic antibody comprises a polypeptide of the following (A), (B) or (C):
(A) a polypeptide comprising an FG loop containing a binding region to a ubiquitin, in which the amino acid sequence of the binding region is represented by the following formula (1):
-Ar-Z-Ar-X-Ar- (1)
wherein Ar represents an aromatic amino acid residue, Z represents a single bond or an amino acid residue other than proline and arginine, and X represents any given amino acid residue,
(B) a polypeptide consisting of an amino acid sequence comprising a deletion, substitution or addition of one or several amino acids with respect to the amino acid sequence of the polypeptide of the above (A) (except for the amino acid sequence represented by the formula (1)), and having binding activity to the ubiquitin, or
(C) a polypeptide having an identity of at least 80% to the amino acid sequence of the polypeptide of the above (A) (except for the amino acid sequence represented by the formula (1)), and having binding activity to the ubiquitin.

2. The synthetic antibody according to claim 1, wherein the amino acid sequence represented by the formula (1) is FWGW (SEQ ID No: 4), YVYGW (SEQ ID No:

5., YWGW (SEQ ID No: 6), YWWYY (SEQ ID No: 7) or YWMW (SEQ ID No: 8).

3. The synthetic antibody according to claim 1, wherein the amino acid sequence of the FG loop is YYGNAFWGWP (SEQ ID No: 16), GHTGGSYVYGWY (SEQ ID No: 17), WYAYSYWGWP (SEQ ID No: 18), EYSYMYWGWP (SEQ ID No: 20), WEISYWWYYQ (SEQ ID No: 21) or YYSGYYWMWY (SEQ ID No: 22).

4. The synthetic antibody according to claim 1, comprising a polypeptide of the following (P1), (P2) or (P3):
(P1) a polypeptide consisting of the amino acid sequence as set forth in any of SEQ ID Nos: 24, 26, 28, 30, 32 and 34,
(P2) a polypeptide consisting of an amino acid sequence comprising a deletion, substitution or addition of one or several amino acids with respect to the amino acid sequence as set forth in any of SEQ ID Nos: 24, 26, 28, 30, 32 and 34 (except for the amino acid sequence of the binding region to the ubiquitin that corresponds to the amino acid sequence represented by the formula (1)), and having binding activity to the ubiquitin, or
(P3) a polypeptide having an identity of at least 80% to the amino acid sequence as set forth in any of SEQ ID Nos: 24, 26, 28, 30, 32 and 34 (except for the amino acid sequence of the binding region to the ubiquitin that corresponds to the amino acid sequence represented by the formula (1)), and having binding activity to the ubiquitin.

5. The synthetic antibody according to claim 1, which has a dissociation constant of 5 nM or less to the ubiquitin.

6. The synthetic antibody according to claim 1, wherein the ubiquitin is a polyubiquitin.

7. DNA encoding a polypeptide of the following (A), (B) or (C):
(A) a polypeptide comprising an FG loop containing a binding region to a ubiquitin, in which the amino acid sequence of the binding region is represented by the following formula (1):
-Ar-Z-Ar-X-Ar- (1)
wherein Ar represents an aromatic amino acid residue, Z represents a single bond or an amino acid residue other than proline and arginine, and X represents any given amino acid residue,
(B) a polypeptide consisting of an amino acid sequence comprising a deletion, substitution or addition of one or several amino acids with respect to the amino acid sequence of the polypeptide of the above (A) (except for the amino acid sequence represented by the formula (1)), and having binding activity to the ubiquitin, or
(C) a polypeptide having an identity of at least 80% to the amino acid sequence of the polypeptide of the above (A) (except for the amino acid sequence represented by the formula (1)), and having binding activity to the ubiquitin.

8. The DNA according to claim 7, wherein the amino acid sequence represented by the formula (1) is FWGW (SEQ ID No: 4), YVYGW (SEQ ID No: 5), YWGW (SEQ ID No: 6), YWWYY (SEQ ID No: 7) or YWMW (SEQ ID No: 8).

9. The DNA according to claim 8, wherein the amino acid sequence of the FG loop is YYGNAFWGWP (SEQ ID No: 16), GHTGGSYVYGWY (SEQ ID No: 17), WYAYSYWGWP (SEQ ID No: 18), EYSYMYWGWP (SEQ ID No: 20), WEISYWWYYQ (SEQ ID No: 21) or YYSGYYWMWY (SEQ ID No: 22).

10. The DNA according to claim 7, encoding a polypeptide of the following (P1), (P2) or (P3):
(P1) a polypeptide consisting of the amino acid sequence as set forth in any of SEQ ID Nos: 24, 26, 28, 30, 32 and 34,
(P2) a polypeptide consisting of an amino acid sequence comprising a deletion, substitution or addition of one or several amino acids with respect to the amino acid sequence as set forth in any of SEQ ID Nos: 24, 26, 28, 30, 32 and 34 (except for the amino acid sequence of the binding region to the ubiquitin that corresponds to the amino acid sequence represented by the formula (1)), and having binding activity to the ubiquitin, or
(P3) a polypeptide having an identity of at least 80% to the amino acid sequence as set forth in any of SEQ ID Nos: 24, 26, 28, 30, 32 and 34 (except for the amino acid sequence of the binding region to the ubiquitin that corresponds to the amino acid sequence represented by the formula (1)), and having binding activity to the ubiquitin.

11. The DNA according to claim 7, which is shown in the following (Q1), (Q2) or (Q3):
(Q1) DNA consisting of the nucleotide sequence as set forth in any of SEQ ID Nos:
23, 25, 27, 29, 31 and 33,
(Q2) DNA hybridizing under stringent conditions with a nucleotide sequence complementary to the nucleotide sequence as set forth in any of SEQ ID Nos: 23, 25, 27, 29, 31 and 33, and having binding activity to the ubiquitin (provided that, among the DNAs hybridizing under the stringent conditions, the nucleotide sequence encoding the amino acid sequence of the binding region to the ubiquitin that corresponds to the amino acid sequence represented by the formula (1) always hybridizes as a codon of the amino acid sequence represented by the formula (1)), or
(Q3) DNA having an identity of at least 80% to the nucleotide sequence as set forth in any of SEQ ID Nos: 23, 25, 27, 29, 31 and 33, and having binding activity to the ubiquitin (provided that, among the DNAs having the identity, the nucleotide sequence encoding the amino acid sequence of the binding region to the ubiquitin that corresponds to the amino acid sequence represented by the formula (1) always encodes the amino acid sequence represented by the formula (1)).

12. An expression vector, comprising the DNA according to claim 7.

13. A transformant, comprising the expression vector according to claim 12.

14. A method for producing a synthetic antibody, which is characterized in that it comprises culturing the transformant according to claim 13, and collecting a synthetic anti-ubiquitin antibody from the obtained culture.

15. A reagent for detecting a ubiquitin or a ubiquitinated substrate, comprising the synthetic antibody according to claim 1.

16. A method for detecting a ubiquitin or a ubiquitinated substrate, which is characterized in that it comprises allowing the synthetic antibody according to claim 1 to come into contact with a test sample.

17. A method for producing a ubiquitin or a ubiquitinated substrate, which is characterized in that it comprises allowing the synthetic antibody according to claim 1 to come into contact with a test sample, and collecting a ubiquitin or a ubiquitinated substrate from a complex of the synthetic antibody and the test sample.

18. The method according to claim 16, wherein the ubiquitin is a polyubiquitin.

19. The method according to claim 17, wherein the ubiquitin is a polyubiquitin.


SEQUENCE LISTING (38 sequences)

SEQ ID NO: 1-3 - skipped (3 entries)

SEQ ID NO: 4 - protein, 4 aa, synthetic construct
  REGION 1..4: note = Synthetic peptide
FWGW

SEQ ID NO: 5 - protein, 5 aa, synthetic construct
  REGION 1..5: note = Synthetic peptide
YVYGW

SEQ ID NO: 6 - protein, 4 aa, synthetic construct
  REGION 1..4: note = Synthetic peptide
YWGW

SEQ ID NO: 7 - protein, 5 aa, synthetic construct
  REGION 1..5: note = Synthetic peptide
YWWYY

SEQ ID NO: 8 - protein, 4 aa, synthetic construct
  REGION 1..4: note = Synthetic peptide
YWMW

SEQ ID NO: 9 - protein, 9 aa, synthetic construct
  REGION 1..9: note = Synthetic peptide
YPYYIIGET

SEQ ID NO: 10 - protein, 10 aa, synthetic construct
  REGION 1..10: note = Synthetic peptide
YYDSGMSKIY

SEQ ID NO: 11 - protein, 10 aa, synthetic construct
  REGION 1..10: note = Synthetic peptide
ASFGFTHMDK

SEQ ID NO: 12 - protein, 10 aa, synthetic construct
  REGION 1..10: note = Synthetic peptide
RQWDKWYVKR

SEQ ID NO: 13 - protein, 10 aa, synthetic construct
  REGION 1..10: note = Synthetic peptide
NIYSVGQGEY

SEQ ID NO: 14 - protein, 10 aa, synthetic construct
  REGION 1..10: note = Synthetic peptide
YTSFSAFGDE

SEQ ID NO: 15 - protein, 10 aa, synthetic construct
  REGION 1..10: note = Synthetic peptide
SSYVTHKEDV

SEQ ID NO: 16 - protein, 10 aa, synthetic construct
  REGION 1..10: note = Synthetic peptide
YYGNAFWGWP

SEQ ID NO: 17 - protein, 12 aa, synthetic construct
  REGION 1..12: note = Synthetic peptide
GHTGGSYVYGWY

SEQ ID NO: 18 - protein, 10 aa, synthetic construct
  REGION 1..10: note = Synthetic peptide
WYAYSYWGWP

SEQ ID NO: 19 - protein, 12 aa, synthetic construct
  REGION 1..12: note = Synthetic peptide
SSKYAGGWIGHE

SEQ ID NO: 20 - protein, 10 aa, synthetic construct
  REGION 1..10: note = Synthetic peptide
EYSYMYWGWP

SEQ ID NO: 21 - protein, 10 aa, synthetic construct
  REGION 1..10: note = Synthetic peptide
WEISYWWYYQ

SEQ ID NO: 22 - protein, 10 aa, synthetic construct
  REGION 1..10: note = Synthetic peptide
YYSGYYWMWY

SEQ ID NO: 23 - DNA, 276 bp, synthetic construct
  misc_feature 1..276: note = Synthetic DNA
  source 1..276: mol_type = other DNA
  CDS 1..276
ctggaagttgtggaagccagcccgacgagcattcagatttcttgggacgcttacccgtactacatcatcggtgaaactgttcgctactatcgcattacctatggcgaaaccggcggtaacagtccggtccaggaatttacggtgccgggttcaaaatcgaccgcgacgatttccggcctgaaaccgggtgttgattataccatcacggtgtacgcagttacctactacggtaacgctttctggggttggccgccgatttctatcaactaccgcacg

SEQ ID NO: 24 - protein, 92 aa, synthetic construct
  REGION 1..92: note = Synthetic polypeptide
LEVVEASPTSIQISWDAYPYYIIGETVRYYRITYGETGGNSPVQEFTVPGSKSTATISGLKPGVDYTITVYAVTYYGNAFWGWPPISINYRT

SEQ ID NO: 25 - DNA, 285 bp, synthetic construct
  misc_feature 1..285: note = Synthetic DNA
  source 1..285: mol_type = other DNA
  CDS 1..285
ctggaagttgtggaagccagcccgacgagcattcagatttcttgggacgcttactacgactctggtatgtctaaaatctacgttcgctactatcgcattacctatggcgaaaccggcggtaacagtccggtccaggaatttacggtgccgggttcaaaatcgaccgcgacgatttccggcctgaaaccgggtgttgattataccatcacggtgtacgcagttaccggtcatactggtggttcttacgtttacggttggtacccgatttctatcaactaccgcacg

SEQ ID NO: 26 - protein, 95 aa, synthetic construct
  REGION 1..95: note = Synthetic polypeptide
LEVVEASPTSIQISWDAYYDSGMSKIYVRYYRITYGETGGNSPVQEFTVPGSKSTATISGLKPGVDYTITVYAVTGHTGGSYVYGWYPISINYRT

SEQ ID NO: 27 - DNA, 279 bp, synthetic construct
  misc_feature 1..279: note = Synthetic DNA
  source 1..279: mol_type = other DNA
  CDS 1..279
ctggaagttgtggaagccagcccgacgagcattcagatttcttgggacgctgcttctttcggtttcactcatatggacaaagttcgctactatcgcattacctatggcgaaaccggcggtaacagtccggtccaggaatttacggtgccgggttcaaaatcgaccgcgacgatttccggcctgaaaccgggtgttgattataccatcacggtgtacgcagttacctggtacgcttactcttactggggttggccgccgatttctatcaactaccgcacg

SEQ ID NO: 28 - protein, 93 aa, synthetic construct
  REGION 1..93: note = Synthetic polypeptide
LEVVEASPTSIQISWDAASFGFTHMDKVRYYRITYGETGGNSPVQEFTVPGSKSTATISGLKPGVDYTITVYAVTWYAYSYWGWPPISINYRT

SEQ ID NO: 29 - DNA, 279 bp, synthetic construct
  misc_feature 1..279: note = Synthetic DNA
  source 1..279: mol_type = other DNA
  CDS 1..279
ctggaagttgtggaagccagcccgacgagcattcagatttcttgggacgctaacatctactctgttggtcagggtgaatacgttcgctactatcgcattacctatggcgaaaccggcggtaacagtccggtccaggaatttacggtgccgggttcaaaatcgaccgcgacgatttccggcctgaaaccgggtgttgattataccatcacggtgtacgcagttaccgaatactcttacatgtactggggttggccgccgatttctatcaactaccgcacg

SEQ ID NO: 30 - protein, 93 aa, synthetic construct
  REGION 1..93: note = Synthetic polypeptide t
LEVVEASPTSIQISWDANIYSVGQGEYVRYYRITYGETGGNSPVQEFTVPGSKSTATISGLKPGVDYTITVYAVTEYSYMYWGWPPISINYRT

SEQ ID NO: 31 - DNA, 279 bp, synthetic construct
  misc_feature 1..279: note = Synthetic DNA
  source 1..279: mol_type = other DNA
  CDS 1..279
ctggaagttgtggaagccagcccgacgagcattcagatttcttgggacgcttacacttctttctctgctttcggtgacgaagttcgctactatcgcattacctatggcgaaaccggcggtaacagtccggtccaggaatttacggtgccgggttcaaaatcgaccgcgacgatttccggcctgaaaccgggtgttgattataccatcacggtgtacgcagttacctgggaaatctcttactggtggtactaccagccgatttctatcaactaccgcacg

SEQ ID NO: 32 - protein, 93 aa, synthetic construct
  REGION 1..93: note = Synthetic polypeptide
LEVVEASPTSIQISWDAYTSFSAFGDEVRYYRITYGETGGNSPVQEFTVPGSKSTATISGLKPGVDYTITVYAVTWEISYWWYYQPISINYRT

SEQ ID NO: 33 - DNA, 279 bp, synthetic construct
  misc_feature 1..279: note = Synthetic DNA
  source 1..279: mol_type = other DNA
  CDS 1..279
ctggaagttgtggaagccagcccgacgagcattcagatttcttgggacgcttcttcttacgttactcataaagaagacgttgttcgctactatcgcattacctatggcgaaaccggcggtaacagtccggtccaggaatttacggtgccgggttcaaaatcgaccgcgacgatttccggcctgaaaccgggtgttgattataccatcacggtgtacgcagttacctactactctggttactactggatgtggtacccgatttctatcaactaccgcacg

SEQ ID NO: 34 - protein, 93 aa, synthetic construct
  REGION 1..93: note = Synthetic polypeptide
LEVVEASPTSIQISWDASSYVTHKEDVVRYYRITYGETGGNSPVQEFTVPGSKSTATISGLKPGVDYTITVYAVTYYSGYYWMWYPISINYRT

SEQ ID NO: 35 - DNA, 291 bp, synthetic construct
  misc_feature 1..291: note = Synthetic DNA
  source 1..291: mol_type = other DNA
  CDS 1..291
ctggaagttgtggaagccagcccgacgagcattcagatttcttgggacgctgcttctttcggtttcactcatatggacaaagttcgctactatcgcattacctatggcgaaaccggcggtaacagtccggtccaggaatttacggtgccgggttcaaaatcgaccgcgacgatttccggcctgaaaccgggtgttgattataccatcacggtgtacgcagttacctggtacgcttactcttactggggttggccgccgatttctatcaactaccgcacggaaattgataaa

SEQ ID NO: 36 - protein, 97 aa, synthetic construct
  REGION 1..97: note = Synthetic polypeptide
LEVVEASPTSIQISWDAASFGFTHMDKVRYYRITYGETGGNSPVQEFTVPGSKSTATISGLKPGVDYTITVYAVTWYAYSYWGWPPISINYRTEIDK

SEQ ID NO: 37 - DNA, 261 bp, Homo sapiens
  source 1..261: mol_type = other DNA
  CDS 1..261
ctggaagttgtggaagccagcccgacgagcattcagatttcttgggacgctccggcggtcacggttcgctactatcgcattacctatggcgaaaccggcggtaacagtccggtccaggaatttacggtgccgggttcaaaatcgaccgcgacgatttccggcctgaaaccgggtgttgattataccatcacggtgtacgcagttaccggtcgtggtgacagcccggccagctctaaaccgatttctatcaactaccgcacg

SEQ ID NO: 38 - protein, 87 aa, Homo sapiens
LEVVEASPTSIQISWDAPAVTVRYYRITYGETGGNSPVQEFTVPGSKSTATISGLKPGVDYTITVYAVTGRGDSPASSKPISINYRT